(19) United States
(12) Patent Application Publication
(10) Pub. No.: US 2021/0330645 A1
(43) Pub. Date: Oct. 28, 2021

(54) ANTIBACTERIAL COMBINATIONS

(71) Applicant: MASSEY UNIVERSITY, Palmerston North (NZ)

(72) Inventors: Jasna Rakonjac, Palmerston North (NZ); Van Hung Vuong Le, Danang City (VN); Catrina Olivera, Lower Hutt (NZ); Raveen Weerasinghe, Oamaru (NZ); Julian Spagnuolo, Basel-Stadt (CH)

(21) Appl. No.: 17/274,115

(22) PCT Filed: Sep. 6, 2019
(86) PCT No.: PCT/IB2019/057511

§ 371 Date: Mar. 5, 2021

(30) Foreign Application Priority Data
Sep. 7, 2018 (AU) 2018903339

(51) Int. Cl. A61K 31/4178; A61K 31/575; A61K 31/345; A61K 31/5377; A61K 31/496; A61K 31/422; A61K 38/14; A61K 8/49; A61K 8/64; A61K 8/63; A61Q 17/00; A01N 47/34; A01N 45/00; A01N 43/76; A01N 43/84; A01N 43/08; A01N 43/50; A01P 1/00; A61P 31/04

(52) U.S. Cl. CPC A61K 31/4178; A61P 31/04; A61K 31/345; A61K 31/5377; A61K 31/496; A61K 31/422; A61K 38/14; A61K 8/49; A61K 8/64; A61K 8/4973; A61K 8/63; A61Q 17/005; A01N 47/34; A01N 45/00; A01N 43/76; A01N 43/84; A01N 43/08; A01N 43/50; A01P 1/00; A61K 31/575

(57) ABSTRACT

Disclosed herein are antibacterial combinations and compositions that are effective against Gram-negative bacteria. Also disclosed are uses of such combinations and compositions for inhibiting the growth and proliferation of Gram-negative bacteria and/or for killing Gram-negative bacteria and/or for treating a Gram-negative bacterial infection.

### FIELD OF THE INVENTION

The present invention relates generally to synergistic antibacterial combinations that inhibit the growth and proliferation of Gram-negative bacteria including Enterobacteria (E. coli, Shigella, Salmonella and Citrobacter). In particular, the present invention relates to synergistic antibacterial combinations of bile salts and nitrofurans, and of bile salts, nitrofurans and glycopeptide antibiotics, to uses of such synergistic combinations to inhibit the growth and/or proliferation of at least one Gram-negative bacterial species, and to methods of using such synergistic combinations to inhibit the growth and/or proliferation of at least one Gram-negative bacterial species.

### BACKGROUND

Antibacterial resistance is one of the most devastating threats to humankind. A recent UK-Prime-Minister-commissioned report chaired by Jim O'Neill (2014) has predicted that Antimicrobial Resistance (AMR) will cause about 10 million deaths per annum, accompanied by a cumulative loss of 60 to 100 trillion USD from the global economy during the period of 2014-2050. The World Health Organization (WHO) has issued a list of 12 bacterial groups for which the new treatments are urgently needed. Among those, the top three (critical) are Gram-negative bacteria: carbapenem-resistant Acinetobacter baumannii, Pseudomonas aeruginosa and Enterobacteriaceae, as well as extended spectrum β-lactamases-(ESBL-)producing Enterobacteriaceae (WHO, 2017). Gram-negative (or double-membrane or diderm) bacteria pose a particular problem due to possession of a highly impermeable outer membrane (absent from Gram-positive or monoderm bacteria such as Staphylococcus or Streptococcus), and an array of multi-drug efflux pumps. Gram-negative pathogens are therefore intrinsically resistant to many existing antibiotics and are associated with a low success rate of antimicrobial development (Iredell et al., 2016, Marston et al., 2016).

There are three major approaches that are used currently to combat multidrug-resistant bacterial pathogens: discovering novel antimicrobials, repurposing drugs already approved for other diseases and employing drug combination therapy. Amongst these approaches, drug combinations are postulated to be capable of enhancement of antibacterial efficacy, deceleration of the rate of resistance and alleviation of side effects such as nephrotoxicity due to a lower concentration of each antibacterial used (Bollenbach, 2015).

One problem with the use of antibiotics against Gram-negative bacteria (e.g. Escherichia coli and Salmonella species) lies in the nature of the cell envelope of these organisms. Gram-negative bacteria are highly resistant to big antibacterials (i.e., molecules above a certain molecular weight). In particular, antibiotics whose molecular weight is over 600 Da cannot cross the Gram negative outer membrane to access targets inside the cell. This is due to the specific structure of the outer layer of the outer membrane (lipopolysaccharide or LPS). Therefore, many antibiotics now used against bacteria that are resistant to carbapenems, β-lactams or quinolones are ineffective against the Gram-negative bacteria due to being larger than 600 Da (e.g. vancomycin, M.W. 1,449 Da). Furthermore, many Gram-negative bacteria (including Enterobacteriaceae) have a wide range of active efflux pumps that remove xenobiotics, including antibiotics and bile salts, from the cells, rendering them recalcitrant to many xenobiotic agents (Paul et al., 2014, Nishino and Yamaguchi, 2001).

Accordingly, there is a need in the art for new drug combinations that are effective against Gram-negative bacterial infections. It is an object of the invention to provide an antibacterial combination, particularly a synergistic antibacterial combination that inhibits the growth and/or proliferation of at least one Gram-negative bacterial species and/or that is a disinfectant that is effective against at least one Gram-negative bacterial species and/or that is effective in treating and/or preventing a bacterial infection, disease or condition in a subject in need thereof that is caused by, or associated with at least one Gram-negative bacterial species, and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF INVENTION

In one aspect the invention relates to an antibacterial combination comprising a nitrofuran and a bile salt. In one embodiment the combination further comprises an antibiotic. In one embodiment the antibiotic is vancomycin (Van). In one embodiment the combination is a synergistic antibacterial combination.

In another aspect the invention relates to a method of inhibiting the growth and/or proliferation of at least one Gram-negative bacterial species, and/or of killing at least one Gram-negative bacterial species comprising contacting the Gram-negative bacterial species with a combination or composition of the invention.

In another aspect the invention relates to the use of an antibacterial combination of the invention for inhibiting the growth and/or proliferation of at least one Gram-negative bacterial species and/or for killing at least one Gram-negative bacterial species.

In another aspect the invention relates to a method of treating a Gram-negative bacterial infection, disease or condition comprising administering a pharmaceutical composition of the invention to a subject in need thereof.

In another aspect the invention relates to an antibacterial combination of the invention, or a pharmaceutical composition of the invention for use in treating a Gram-negative bacterial infection, disease and/or condition.

In another aspect the invention relates to the use of an antibacterial combination of the invention in the manufacture of a medicament for treating a Gram-negative bacterial infection, disease and/or condition.

In another aspect the invention relates to the use of an antibacterial combination of the invention to make a cosmetic composition.

Various embodiments of the different aspects of the invention as discussed above are also set out below in the detailed description of the invention, but the invention is not limited thereto.

Other aspects of the invention may become apparent from the following description which is given by way of example only and with reference to the accompanying drawings.

### BRIEF DESCRIPTION OF DRAWINGS

The invention will now be described by way of example only and with reference to the drawings in which:

FIG. 1 Structural formulae of nitrofurans: A) Furazolidone (FZ); B) Nitrofurantoin (NF); C) Nitrofurazone (NFZ); D) CM4, Pubchem ID AC1LGLMG (no CAS number). Chemical name: N′-[(5-nitrofuran-2-yl)methylidene]furan-2-carbohydrazide or N-[(5-nitrofuran-2-yl)methylideneamino]furan-2-carboxamide.

FIG. 2 Structural formulae of bile salts: A, Unconjugated bile salts; B, glycine conjugated bile salt; C, taurine conjugated bile salt; D, Annotation of R¹ and R² in the formulae.

FIG. 3 Structural formula of glycopeptide antibiotic vancomycin (Van).

FIG. 4 Three-way interaction of Van, FZ and DOC in growth inhibition of E. coli K12. Datapoints in the graphs are concentrations (A) or Fractional Inhibitory Concentration index (FIC) values (B) that caused 90% growth inhibition for combinations (or each molecule alone; as indicated in the graph).

FIG. 5 Three-way interaction of Van, FZ and DOC in growth inhibition of Escherichia coli O157. Datapoints in the graphs are concentrations (A) or FIC values (B) that caused 90% growth inhibition for combinations (or each molecule alone; as indicated in the graph).

FIG. 6 Interactions of four nitrofurans with DOC in growth inhibition of an E. coli UTI isolate. Graphs (isobolograms) are obtained using a checkerboard analysis at multiple concentration of molecules. A-D, each data point represents the minimum molecule concentrations alone or in combination causing 90% inhibition to bacterial growth. E, each data point corresponds to the FIC (ratios of the 90% growth inhibition concentrations in combination vs. alone) for each of the analysed nitrofurans; denoted as 5-Nitrofurans (y axis) and DOC (x axis).

FIG. 7 Interactions of four nitrofurans with DOC in growth inhibition of E. coli O157. Graphs (isobolograms) are obtained using a checkerboard analysis at multiple concentration of molecules. A-D, each data point represents the minimum molecule concentrations alone or in combination causing 90% inhibition to bacterial growth. E, each data point corresponds to the FIC (ratios of the 90% growth inhibition concentrations in combination vs. alone) for each of the analysed nitrofurans; denoted as 5-Nitrofurans (y axis) and DOC (x axis).

FIG. 8 Interactions of four nitrofurans with DOC in growth inhibition of E. coli K12. Graphs (isobolograms) are obtained using a checkerboard analysis at multiple concentration of molecules. A-D, each data point represents the minimum molecule concentrations alone or in combination causing 90% inhibition to bacterial growth. E, each data point corresponds to the FIC (ratios of the 90% growth inhibition concentrations in combination vs. alone) for each of the analysed nitrofurans; denoted as 5-Nitrofurans (y axis) and DOC (x axis).

FIG. 9 Interactions of four nitrofurans with DOC in growth inhibition of Salmonella enterica sv. typhimurium LT2. Graphs (isobolograms) are obtained using a checkerboard analysis at multiple concentration of molecules. A-D, each data point represents the minimum molecule concentrations alone or in combination causing 90% inhibition to bacterial growth. E, each data point corresponds to the FIC (ratios of the 90% growth inhibition concentrations in combination vs. alone) for each of the analysed nitrofurans; denoted as 5-Nitrofurans (y axis) and DOC (x axis).

FIG. 10 Interactions of four nitrofurans with DOC in growth inhibition of Citrobacter gillenii. Graphs (isobolograms) are obtained using a checkerboard analysis at multiple concentration of molecules. A-D, each data point represents the minimum molecule concentrations alone or in combination causing 90% inhibition to bacterial growth. E, each data point corresponds to the FIC (ratios of the 90% growth inhibition concentrations in combination vs. alone) for each of the analysed nitrofurans; denoted as 5-Nitrofurans (y axis) and DOC (x axis).

FIG. 11 Interactions of nitrofurans with DOC in growth inhibition of Klebsiella pneumoniae. Graphs (isobolograms) are obtained using a checkerboard analysis at multiple concentration of molecules. A-C, Each data point represents the minimum molecule concentrations, alone or in combination, causing 90% inhibition of bacterial growth (y axis) for FZ (A), NF (B) or NFZ (C) and DOC (x axis).

FIG. 12 Interaction of FZ and DOC in growth inhibition of ampicillin- and streptomycin-resistant E. coli K12. Graphs (isobolograms) are obtained using a checkerboard analysis at multiple concentration of molecules. A and B, strain K1508 (ampicillin-sensitive, streptomycin-resistant); C and D, strain K2524 (ampicillin-resistant, streptomycin-resistant; see Table 1 for genotypes). A; C, each data point represents the minimum molecule concentrations alone or in combination causing 90% inhibition to bacterial growth. B; D, each data point corresponds to the FIC (ratios of the 90% growth inhibition concentrations in combination vs. alone) for FZ (y axis) and DOC (x axis).

FIG. 13 Time-kill analysis of the DOC and FZ combination in killing S. typhimurium strain LT2. The data is presented as the mean±standard error of the mean (SEM) of three independent measurements. The count of the live S. typhimurium was determined at indicated time points by titration of colony-forming units on agar plates. The low limit of detection was 60 cfu/mL.

FIG. 14 Time-kill analysis of the DOC and FZ combination in killing E. coli K12 laboratory strain K1508. The data is presented as the mean±standard error of the mean (SEM) of three independent measurements. The count of the live E. coli was determined at indicated time points by titration of colony-forming units on agar plates. The lower limit of detection was 60 cfu/mL.

FIG. 15 Time-kill analysis of the triple DOC, FZ and VAN combination in killing E. coli strain K1508. The data is presented as the mean±standard error of the mean (SEM) of three independent measurements. The count of the live E. coli was determined at indicated time points by titration of colony-forming units on agar plates. The lower limit of detection was 60 cfu/mL.

FIG. 16 Effect of the ΔtolC and ΔacrA mutations on FZ-DOC synergy in E. coli. Isobolograms characterising the interactions of FZ and DOC in growth inhibition assays of the E. coli K12 strain K1508 (WT or wild-type; A), and two isogenic deletion mutants, K2424 (ΔacrA; B); K2403 (ΔtolC; C). Each data point represents a minimum drug concentrations alone or in combination causing 90% inhibition to bacterial growth. D, Isobolograms of all three strains; each data point corresponds to the FIC (ratios of the 90% growth inhibition concentrations in combination vs. alone) for FZ (y axis) and DOC (x axis).

FIG. 17 Effect of the ΔtolC and ΔacrA mutations on DOC synergy with NF, NFZ and CM4 in E. coli. Isobolograms characterising interactions of DOC with NF (A), NFZ (B) and CM4 (C) in growth inhibition assays of the E. coli K12 strain K1508 (WT or wild-type) and two isogenic deletion mutants, ΔacrA and ΔtolC). Each data point corresponds to the FIC (ratios of the 90% growth inhibition concentrations in combination vs. alone) for one of the three nitrofurans (y axis) and DOC (x axis).

FIG. 18 Recovery of FZ-DOC synergy in complemented ΔtolC and ΔacrA mutants. Isobolograms of FZ-DOC interactions in growth inhibition of: A, ΔtolC mutant (ΔtolC) and a derived strain containing a plasmid expressing to/C gene (ΔtolC+tolC); B, ΔacrA mutant (ΔacrA) and a derived strain containing a plasmid expressing acrA gene and (ΔacrA+acrA). Each data point corresponds to the FIC (ratios of the 90% growth inhibition concentrations in combination vs. alone) for FZ (y axis) and DOC (x axis).

FIG. 19 Effect of the hmp gene overexpression on FZ-DOC synergy. The isobologram of DOC and FZ interaction in E. coli having differential expression of NO-detoxifying protein Hmp. WT, strain E. coli laboratory strain K1508; WT+hmp, K1508 containing a plasmid (pCA24N) expressing hmp gene under the control of a T5-lac hybrid promoter was induced by IPTG (1 mM). Each data point corresponds to the FIC (ratios of the 90% growth inhibition concentrations in combination vs. alone) for FZ (y axis) and DOC (x axis).

FIG. 20 Combined DOC and FZ treatment of E. coli-inoculated meat slices. Solution containing 2,500 μg/ml of DOC and 0.32 μg/ml of FZ was applied to E. coli-K12-inoculated meat surface and bacterial titers were monitored after 10 min incubation at room temperature (A); After 2 h incubation at 30° C. (B). Pre-treatment, titer before applying antibacterial solution; PBS treatment, phosphate buffer saline (pH 7.4) buffer without antibacterials; DOC/FZ treatment, 2,500 μg/ml of DOC and 0.32 μg/ml of FZ in PBS. The titer values are presented as mean±SEM of triplicate.

FIG. 21. Combined DOC and FZ treatment of E. coli-inoculated cow hide. Solution containing 2,500 μg/mL of DOC and 0.32 μg/ml of FZ was applied to E. coli-K12-inoculated hide surface and bacterial titres were monitored after 6 h incubation at 30° C. Pre-treatment, titre before applying antibacterial solution; PBS treatment, phosphate buffer saline (pH 7.4) without antibacterials; DOC/FZ treatment, 2,500 μg/ml of DOC and 0.32 μg/ml of FZ in water. The titre values are presented as mean±SEM of triplicate.

FIG. 22 CM4 interactions with vancomycin (Van). Dose-response plots comparing % growth inhibition at increasing CM4 concentrations in the presence or absence of Van (75 μg/mL). A, E. coli O157; B, E. coli UTI isolate; C, Salmonella enterica SA223a; D, Citrobacter gillenii. Concentration of Van is 75 μg/mL.

FIG. 23 Nitrofurantoin (NF) interactions with vancomycin (Van) in growth inhibition of E. coli. Dose-response plots comparing % growth inhibition at increasing CM4 concentrations in the presence or absence of Van (75 μg/mL). A, E. coli O157; B, E. coli UTI isolate.

FIG. 24 CM4 interaction with Van in growth inhibition of E. coli O157. Graphs (isobolograms) comparing inhibition by combinations vs. alone. A; each data point represents the minimum molecule concentrations alone or in combination causing 90% inhibition to bacterial growth. B, each data point corresponds to the FIC (ratios of the 90% growth inhibition concentrations in combination vs. alone).

FIG. 25 FZ interaction with Van in growth inhibition of E. coli. A, O157; B, two K12 laboratory strains. Graphs (isobolograms) comparing inhibition by combinations vs. alone. Each data point corresponds to the FIC (ratios of the 90% growth inhibition concentrations in combination vs. alone).

FIG. 26 Time-kill experiment using FZ-Van combination. Points were derived from a single experiment. The count of the live E. coli was determined at indicated time points by titration of colony-forming units on agar plates. The lower limit of detection was 100 cfu/mL.

FIG. 27 Three-way interaction of Van, FZ and DOC in growth inhibition of Salmonella typhimurium LT2. Datapoints in the graphs are concentrations (A) or FIC values (B) that caused 90% growth inhibition for combinations (or each molecule alone; as indicated in the graph).

FIG. 28 Three-way interaction of Van, FZ and DOC in growth inhibition of Citrobacter gillenii. Datapoints in the graphs are concentrations (A) or FIC values (B) that caused 90% growth inhibition for combinations (or each molecule alone; as indicated in the graph).

FIG. 29 Three-way interaction of Van, FZ and DOC in growth inhibition of E. coli UTI isolate. Datapoints in the graphs are concentrations (A) or FIC values (B) that caused 90% growth inhibition for combinations (or each molecule alone; as indicated in the graph).

FIG. 30 Three-way interaction of Van, NF and DOC in growth inhibition of E. coli UTI isolate. Datapoints in the graphs are concentrations (A) or FIC values (B) that caused 90% growth inhibition for combinations (or each molecule alone; as indicated in the graph).

FIG. 31 Three-way interaction of Van, NFZ and DOC in growth inhibition of E. coli K12. Datapoints in the graphs are concentrations (A) or FIC values (B) that caused 90% growth inhibition for combinations (or each molecule alone; as indicated in the graph).

FIG. 32 Three-way interaction of Van, CM4 and DOC in growth inhibition of E. coli K12. Datapoints in the graphs are concentrations (A) or FIC values (B) that caused 90% growth inhibition for combinations (or each molecule alone; as indicated in the graph).

### DETAILED DESCRIPTION OF THE INVENTION

### Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains. Examples of definitions of common terms in microbiology, molecular biology, pharmacology and biochemistry can be found in (Meyers, 1995, Lewin et al., 2011, Madigan et al., 2009, Singleton and Sainsbury, 2006, Lederberg, 2000, Reddy, 2007).

It is also believed that practice of the present invention can be performed using standard microbiological, molecular biology, pharmacology and biochemistry protocols and procedures as known in the art, and as described, for example in (Burtis et al., 2015, Lewin et al., 2011, Whitby and Whitby, 1993, Reddy, 2007, Sambrook and Russell, 2001) and other commonly available reference materials relevant in the art to which this disclosure pertains, and which are all incorporated by reference herein in their entireties.

The following definitions are presented to better define the present invention and as a guide for those of ordinary skill in the art in the practice of the present invention.

As used herein an “antibacterial combination” means a combination of at least 2 of a bile salt, a nitrofuran and an antibiotic that inhibits the growth and/or proliferation of at least one Gram-negative bacterial species, and/or that kills at least one Gram-negative bacterial species.

In the context of the present disclosure, “inhibiting the growth and/or proliferation” of at least one Gram-negative bacterial species refers to no detectable increase in the number of bacteria present, and/or in the duration of the bacterial presence or infection under the conditions that otherwise stimulate bacterial multiplication (in the absence of the antibacterial combination).

In some embodiments, “inhibiting the growth and/or proliferation” of at least one Gram-negative bacterial species is determined by comparative assay of the optical density at 600 nm over time, of a Gram-negative bacterial control culture vs. a Gram-negative bacterial culture treated with an antibacterial combination or composition as described herein. In some embodiments, inhibition is observed when the optical density of the treated culture is less than 10% of the optical density relative to the control culture.

In the context of the present disclosure, “killing” of bacteria refers to decrease in the number of viable Gram-negative bacterial cells remaining in a population of Gram-negative bacterial cells exposed to an antibacterial combination as described herein as compared to the number of viable Gram-negative bacterial cells in an untreated population.

In some embodiments, “killing” of Gram-negative bacteria is determined by measuring decrease in the number of viable bacterial cells at set time points during culturing in the presence of antibacterial combinations (“time-kill curve”)

The phrase “a Gram-negative bacterial infection, disease or condition” as used herein refers to any bacterial infection, disease or condition that is caused by or associated with a particular species of Gram-negative bacteria.

In the context of the present disclosure, the Fractional Inhibitory Concentration index (FICi) for any two drugs (e.g. A and B) is calculated as follows: FICi=FIC_{A}+FIC_{B}, where FIC of each drug is calculated as Minimal Inhibitory Concentration_{Acomb}/Minimal Inhibitory Concentration_{Aalone} (MIC_{Acomb}/MIC_{Aalone}) (i.e. the ratio of 90% growth inhibition when applied in combination vs. alone.) (Doern, 2014)

In the context of the present disclosure, a “synergistic effect” is demonstrated when the minimal FICi (at a combination of concentrations where the sum of their FIC's is the lowest) is 0.5 or less for double combinations.

As used herein, the terms “treat”, “treating” and “treatment” refer to therapeutic measures which reduce, alleviate, ameliorate, manage, prevent, restrain, stop or reverse bacterial infection caused by or associated with Gram-negative bacterial species, including the symptoms associated with or related to such a bacterial infection. The subject may show observable or measurable (statistically significant) decrease in one or more of the symptoms associated with or related bacterial infection as known to those skilled in the art, as indicating improvement.

The term “effective amount” as used herein means an amount effective to protect against, delay, reduce, stabilize, improve or treat a bacterial infection, disease and/or condition as known in the art, and/or as described herein. In particular, an “therapeutically effective amount” of an anti-microbial combination as described is an amount that is sufficient to achieve at least a lessening of the symptoms associated with a bacterial infection that is being or is to be treated or that is sufficient to achieve a reduction in bacterial growth, or that is sufficient to increase in bacterial susceptibility to other therapeutic agents or natural immune clearance.

In some embodiments, an effective amount is an amount sufficient to achieve a statistically different result as compared to an untreated control.

The term “about” when used in connection with a referenced numeric indication means the referenced numeric indication plus or minus up to 10% of that referenced numeric indication. For example, “about 100” means from 90 to 110 and “about six” means from 5.4 to 6.6.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

The term “consisting” essentially or as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### Detailed Description

Antibiotic resistance in Gram-negative enterobacteria poses a serious threat to global health care. In particular, many Gram-negative bacteria are resistant to the first-line defense antibiotics, such as β-lactams (penicillins; cephalosporins and derivatives thereof), trimethoprim and quinolones. These resistant microbes top the 2017 WHO list of organisms against which novel antimicrobials are required.

Described herein are the inventor's findings that certain combinations comprising a nitrofuran and a bile salt can inhibit the growth and/or proliferation of certain Gram-negative bacterial species, demonstrating surprising and synergistic bactericidal and/or bacteriostatic effects. These combinations do not include antibiotics to which antibiotic resistance has emerged to date. The inventors believe that they are the first to show that a nitrofuran and a bile salt can interact synergistically as an effective antibacterial combination for killing Enterobacteria. The inventors have also surprisingly identified a particularly advantageous effect of the antibacterial combinations described herein where the concentrations of each compound included in the combination is below the concentration that would associated with toxicity in a mammal. Without wishing to be bound by theory, the inventors believe that the mechanism of this synergy relates to the ability of nitrofurans to inhibit Gram negative efflux pumps that expel bile salts.

Gram-negative bacteria are much more recalcitrant to a number of antimicrobials than are Gram-positive bacteria, due to the poor permeability of the lipopolysaccharide (LPS), obstructing the access of antimicrobials to their targets (Silver, 2011). The size of the antimicrobials that are effective against Gram-negative bacteria is limited by the size of proteinaceous channels and pores that puncture the outer membrane. The majority of these are non-specific channels termed porins that restrict the size of molecules that cross this barrier to 600 Da (Silver, 2011). This excludes a number of antibiotics whose molecular weight is larger than this cut-off molecular weight, such as vancomycin, bacitracin, linezolid, daptomycin, novobiocin and others. If at all, >600 Da antibiotics inhibit growth of Gram-negative bacteria at very high (typically nephrotoxic) concentrations (Mergenhagen and Borton, 2014).

Another strategy applied by Gram-negative bacteria against antimicrobials is pumping of xenobiotic molecules out of the cell using a wide range of active efflux pumps that traverse the envelope (inner membrane, periplasm and outer membrane), rendering Gram-negative bacteria recalcitrant to many xenobiotic agents, such as DOC. The tripartite Nodulation-Division-Resistance (NDR) AcrAB-TolC system that is comprised of a channel in the outer membrane (TolC), a periplasmic protein adaptor (AcrA) and an inner membrane efflux protein (AcrB) is the dominant efflux pump that expulses DOC from E. coli (Paul et al., 2014, Nishino and Yamaguchi, 2001).

The inventors have determined that certain combinations of nitrofurans with bile salts have bacteriostatic and/or bactericidal effects against Gram-negative enterobacteria. In some embodiments the enterobacteria are selected from E. coli, S. typhimurium and Citrobacter gillenii.

Also, the inventors have determined that the combination of a nitrofuran, a bile salt and a glycopeptide antibiotic, particularly vancomycin, interacts synergistically to provide an effective triple Gram-negative antibacterial combination. Particularly advantageous is that such an antibacterial combination comprises a concentration of each individual component that is below the concentration associated with the mammalian toxicity of the component if used alone. For example, the recommended therapeutic concentration of vancomycin that is considered to be below nephrotoxic level in a 6-day treatment is below 20 μg/mL, whereas MIC for E. coli is 250 μg/mL when used on its own (Elyasi et al., 2012, Mergenhagen and Borton, 2014).

Besides decreased toxicity to patients, the use of the Gram-negative antibacterial combinations as described herein is expected to decrease the frequency of resistant mutations arising in populations of targeted bacteria, as the chance of a bacterium containing at least three mutations that would be required to develop effective resistance is a product of individual mutation rates which are each ˜10⁻⁶, resulting in a much smaller probability for triple-antibacterial combination (˜10⁻¹⁸).

E. coli, S. typhimurium and Citrobacter gillenii are enterobacteria, some of which are major carriers of antimicrobial resistance genes (AMR) genes against β-lactam antibiotics (penicillins, cephalosporins and derivatives thereof). The three components of the antibacterial combinations described herein are different in their chemical structures, mechanisms of action and/or bacterial targets than the antibiotics affected by AMR identified in Gram-negative bacteria. Therefore, the antibacterial combinations described herein provide a solution to the lack of effective treatments for infections by Gram-negative bacteria carrying major widespread AMR genes, e.g. against extended spectrum β-lactamase-(ESBL-) producing Enterobacteriaceae (WHO, 2017), which top the list of bacteria that require urgent antibiotic development.

In Examples 1-6, E. coli strains are used as a model organism to show the synergy of certain nitrofurans (“CM4”, nitrofurantoin, nitrofurazon and furazolidone) with certain bile salts (sodium deoxycholate). S. enterica sv. typhimurium and Citrobacter gillenii were also used to demonstrate the synergy of these antibacterial combinations.

Based on the findings described herein, including in the examples, the inventors believe that antibacterial combinations comprising at least one nitrofuran and at least one bile salt as described herein can be used to inhibit the growth and/or proliferation of many different bacteria, particularly enterobacteria, preferably Escherichia spp., Salmonella spp. and/or Citrobacter spp., preferably E. coli, S. typhimurium and/or Citrobacter gillenii.

The inventors have also determined that a combination of a nitrofuran, a bile salt and a glycopeptide antibiotic acts synergistically, and is effective at inhibiting the growth and/or proliferation of Gram-negative bacteria or for killing Gram-negative bacteria, where the concentration of each compound in the combination is below the concentration of that compound that would be required for growth inhibition if used alone. Furthermore, the growth-inhibitory concentration of the glycopeptide antibiotic would be toxic in a mammal were the compound to be used alone. Specifically, the inventors have found that a combination of a nitrofuran with sodium deoxycholate and vancomycin, inhibits growth of E. coli, where vancomycin is at sub-toxic concentrations [below 20 μg/mL; (Elyasi et al., 2012, Mergenhagen and Borton, 2014)]. Concentration of vancomycin required for E. coli growth inhibition when used on its own is >250 μg/mL. The inventors have surprisingly found that these combinations lead to major lowering of individual minimal inhibitory concentrations for E. coli as a model Gram-negative pathogen. The inventors believe that this is the first time that the triple combination of a nitrofuran, a bile salt and a glycopeptide antibiotic, particularly vancomycin, has been identified for inhibition of Gram-negative bacteria. Without wishing to be bound by theory, the inventors believe that because the chemical structures, targets and mechanisms of action of the components in the antibacterial combinations as described herein are different from the antibiotics against which the recent global antimicrobial-resistance (AMR) in Gram-negative bacteria has emerged, the antibacterial combinations described herein will provide an effective therapy against these AMR Gram-negative pathogens.

Accordingly, in one aspect the invention relates to an antibacterial combination comprising a nitrofuran and a bile salt.

In one embodiment the combination is a synergistic combination.

In one embodiment the combination is a Gram-negative antibacterial combination.

In one embodiment the nitrofuran comprises a 5-nitrofuran ring.

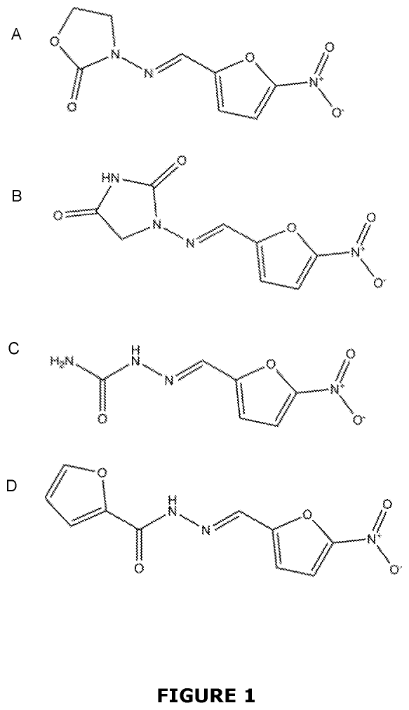

In one embodiment the nitrofuran comprises a structure as shown in FIG. 1.

Structures of three nitrofurans used as models for demonstrating the synergies of various antibacterial combinations of the invention (as in the examples) are provided in FIGS. 1A, B, C and D. Furazolidone (FZ) (FIG. 1A), nitrofurantoin (NF) (FIG. 1B) and nitrofurazone (NFZ) (FIG. 1C) are prescription medicines used primarily as antibacterial/anthelmintic drugs. The fourth nitrofuran (FIG. 1D) termed herein as “CM4”, is only available as a “for research only” compound and is sold within small-molecule libraries. The PubChem ID of CM4 is AC1LGLMG; chemical name: N′-[(5-nitrofuran-2-yl)methylidene]furan-2-carbohydrazide or N-[(5-nitrofuran-2-yl)methylideneamino]furan-2-carboxamide.

Furazolidone (FZ) is a 5-nitrofuran-derived antimicrobial agent (3-[(E)-(5-nitrofuran-2-yl)methylideneamino]-1,3-oxazolidin-2-one) which was developed in the late 1940s. This drug is used to treat bacterial diarrhea, giardiasis and is sometimes included as a component in Helicobacter pylori treatment (Petri, 2005, Hajaghamohammadi et al., 2014). This drug and other 5-nitrofuran compounds, such as nitrofurantoin (NF) and nitrofurazone (NFZ), are the prodrugs which require reductive activation catalysed by two type-I oxygen-insensitive nitroreductases, NfsA and NfsB, in a redundant manner. These two enzymes perform stepwise 2-electron reduction of the nitro moiety of the compound into the nitroso and the hydroxylamino intermediates and a biologically inactive amino-substituted product (Sandegren et al., 2008). The detailed mechanism of how bacterial cells are killed by the reactive intermediate is yet to be clarified. Nevertheless, it has been proposed that the hydroxylamino derivatives could trigger DNA lesions, disrupt protein structure and arrest protein biosynthesis (McOsker and Fitzpatrick, 1994, Bertenyi and Lambert, 1996, Roldan et al., 2008, Ona et al., 2009). Some reports also suggested that during the activation process nitric oxide (NO) could be generated, inhibiting the electron transport chain of bacterial cells. However, clear evidence for NO production from furazolidone activation is not available as yet (Vumma et al., 2016).

In one embodiment the nitrofuran is selected from the group consisting of CM4, difurazone, furazolidone; nifurfoline, nifuroxazide, nifurquinazol, nifurtoinol, nifurzide, nitrofural (nitrofurazone), nitrofurantoin, ranbezolid and nifuratel, preferably CM4, furazolidone or nitrofurantoin. Preferably the nitrofuran is CM4, furazolidone, nitrofurazone or nitrofurantoin.

In one embodiment the concentration of the nitrofuran present the antibacterial combination is from about 0.1 μg/mL to about 2 μg/mL, preferably from about 0.3 μg/mL to about 1.3 μg/mL, preferably from about 0.5 μg/mL to about 0.8 μg/mL, preferably from about 0.6 μg/mL to about 0.7 μg/mL, preferably about 0.625 μg/mL, preferably about 0.5 μg/mL.

In one embodiment the concentration of the nitrofuran present the antibacterial combination is from 0.1 μg/mL to 2 μg/mL, preferably from 0.3 μg/mL to 1.3 μg/mL, preferably from 0.5 μg/mL to 0.8 μg/mL, preferably from 0.6 μg/mL to 0.7 μg/mL, preferably 0.625 μg/mL, preferably about 0.5 μg/mL.

In one embodiment the concentration of the nitrofuran present the antibacterial combination is from about 0.1 μg/mL to about 1 μg/mL, preferably from about 0.2 μg/mL to about 0.5 μg/mL, preferably from about 0.3 μg/mL to about 0.4 μg/mL, preferably about 0.325 μg/mL.

In one embodiment the concentration of the nitrofuran present the antibacterial combination is from 0.1 μg/mL to 1 μg/mL, preferably from 0.2 μg/mL to 0.5 μg/mL, preferably from 0.3 μg/mL to 0.4 μg/mL, preferably 0.325 μg/mL.

In one embodiment the nitrofuran is furazolidone (FZ) and the concentration present in the antibacterial combination is from about 0.1 to about 1 μg/mL, preferably about 0.25 to about 0.75 μg/mL, preferably about 0.5 μg/mL.

In one embodiment the nitrofuran is FZ and the concentration present in the antibacterial combination is from 0.1 to 1 μg/mL, preferably 0.25 to 0.75 μg/mL, preferably 0.5 μg/mL.

In one embodiment the nitrofuran is nitrofurantoin (NF) and the concentration present the antibacterial combination is from about 0.1 μg/mL to about 100 μg/mL, preferably from about 0.5 μg/mL to about 50 μg/mL, preferably from about 1 μg/mL to about 15 μg/mL, preferably about 5 to about 10 μg/mL, preferably about 8 μg/mL.

In one embodiment the nitrofuran is nitrofurantoin (NF) and the concentration present the antibacterial combination is from 0.1 μg/mL to 100 μg/mL, preferably from 0.5 μg/mL to 50 μg/mL, preferably from 1 μg/mL to 15 μg/mL, preferably 5 to 10 μg/mL, preferably 8 μg/mL.

In one embodiment the nitrofuran is NF and the concentration present the antibacterial combination is from about 0.1 μg/mL to about 100 μg/mL, preferably from about 0.5 μg/mL to about 50 μg/mL, preferably from about 1 μg/mL to about 15 μg/mL, preferably about 5 to about 10 μg/mL, preferably about 8 μg/mL.

In one embodiment the nitrofuran is nitrofurazone (NFZ) and the concentration present the antibacterial combination is from 0.1 μg/mL to 100 μg/mL, preferably from 0.5 μg/mL to 50 μg/mL, preferably from 0.75 μg/mL to 10 μg/mL, preferably 1 to 5 μg/mL, preferably 2 μg/mL.

In one embodiment the nitrofuran is NFZ and the concentration present the antibacterial combination is from about 0.1 μg/mL to about 100 μg/mL, preferably from about 0.5 μg/mL to about 50 μg/mL, preferably from about 0.75 μg/mL to about 10 μg/mL, preferably about 1 to about 5 μg/mL, preferably about 2 μg/mL.

In one embodiment the nitrofuran is CM4 and the concentration present the antibacterial combination is from 0.1 μg/mL to 100 μg/mL, preferably from 0.5 μg/mL to 50 μg/mL, preferably from 1 μg/mL to 15 μg/mL, preferably 5 to 10 μg/mL, preferably 8 μg/mL.

In one embodiment the nitrofuran is CM4 and the concentration present the antibacterial combination is from about 0.1 μg/mL to about 100 μg/mL, preferably from about 0.5 μg/mL to about 50 μg/mL, preferably from about 1 μg/mL to about 15 μg/mL, preferably about 5 to about 10 μg/mL, preferably about 8 μg/mL.

Bile salts are a component of bile which is secreted into duodenum to support the fat digestion, regulate glucose homeostasis, modulate inflammatory processes and confer some antibacterial protection (Faustino et al., 2016).

In one embodiment the bile salt is a sterol-derived facial amphipathic compound in bile.

In one embodiment the bile salts are mammalian bile salts that comprise primary bile salts, cholate and chenodeoxycholate, and the secondary bile salts, including deoxycholate, lithocholate, and ursodeoxycholate (Begley et al., 2005, Faustino et al., 2016). The bile salts exist in conjugated form with amino acid glycine or taurine or in unconjugated form upon the microbial bile salt hydrolase.

In terms of antibacterial action, bile salts have been reported to attack different cellular sites including disrupting the cell membrane, causing DNA damage and triggering protein aggregation (Merritt and Donaldson, 2009, Cremers et al., 2014).

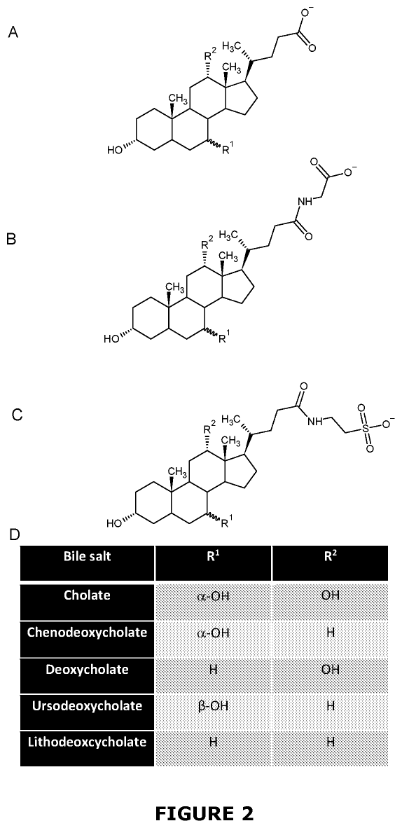

Sodium Deoxycholate (DOC, sodium; (4R)-4-[(3R,5R,8R,9S,10S,12S,13R,14S,17R)-3,12-dihydroxy-10,13-dimethyl-2,3,4,5,6,7,8,9,11,12,14,15,16,17-tetradecahydro-1H-cyclopenta[a]phenanthren-17-yl]pentanoate) is a facial amphipathic compound in bile, which is secreted into the duodenum to aid lipid digestion due to its surfactant properties; it also confers some antimicrobial protection (Begley et al., 2005). E. coli, as typical of Gram-negative bacterium, is highly resistant to DOC thanks to restricting the intracellular accumulation of DOC by employment of diverse active efflux pumps and down-regulation of outer membrane porins or mitigation of DOC-mediated toxic effects by activation of various stress responses (Nishino and Yamaguchi, 2001, Merritt and Donaldson, 2009, Paul et al., 2014). The structure of sodium deoxycholate, a bile salt that was used in an antibacterial combination as described herein is shown in FIG. 2A. Deoxycholate is one of the natural bile salts found in mammalian digestive tract.

In one embodiment the bile salt or functional analogue or derivative thereof is selected from the group consisting of deoxycholate, cholate, chenodeoxycholate, taurocholate, glycocholate, taurochenodeoxycholate, glycochenodeoxycholate, lithocholate, and ursodeoxycholate.

In one embodiment the bile salt or functional analogue or derivative thereof is sodium deoxycholate (DOC).

In one embodiment the concentration of DOC present the antibacterial combination is from about 500 μg/mL to about 5000 μg/mL, preferably from about 1000 μg/mL to about 4000 μg/mL, preferably from about 1500 μg/mL to about 3500 μg/mL, preferably from about 2000 μg/mL to about 3000 μg/mL, preferably from about 2250 μg/mL to about 2750 μg/mL, preferably 2500 μg/mL.

In one embodiment the concentration of DOC present the antibacterial combination is from 500 μg/mL to 5000 μg/mL, preferably from 1000 μg/mL to 4000 μg/mL, preferably from 1500 μg/mL to 3500 μg/mL, preferably from 2000 μg/mL to 3000 μg/mL, preferably from 2250 μg/mL to 2750 μg/mL, preferably 2500 μg/mL.

In one embodiment the concentration of DOC present the antibacterial combination is from about 250 μg/mL to about 2500 μg/mL, preferably from about 500 μg/mL to about 2250 μg/mL, preferably from about 750 μg/mL to about 2000 μg/mL, preferably from about 1000 μg/mL to about 1750 μg/mL, preferably from about 1200 μg/mL to about 1600 μg/mL, preferably about 1562 μg/mL, preferably about 1250 μg/mL, preferably 625 μg/mL.

In one embodiment the concentration of DOC present the antibacterial combination is from 250 μg/mL to 2500 μg/mL, preferably from 500 μg/mL to 2250 μg/mL, preferably from 750 μg/mL to 2000 μg/mL, preferably from 1000 μg/mL to 1750 μg/mL, preferably from 1200 μg/mL to 1600 μg/mL, preferably 1562 μg/mL, preferably 1250 μg/mL, preferably 625 μg/mL.

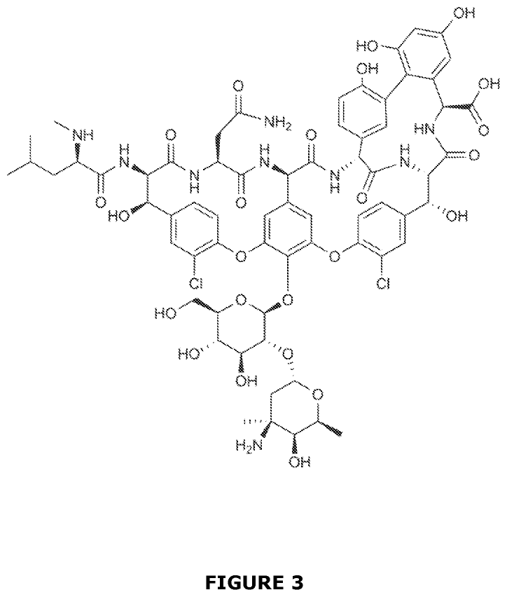

In one embodiment, the antibacterial combination further comprises an antibiotic. In one embodiment the antibiotic is a glycopeptide antibiotic. In one embodiment the glycopeptide antibiotic is vancomycin (FIG. 3) or a functional analogue or derivative thereof.

A glycopeptide antibiotic is a drug of microbial origin comprised of glycosylated cyclic or polycyclic non-ribosomal peptides. Significant glycopeptide antibiotics include vancomycin, teicoplanin, telavancin, ramoplanin and decaplanin.

In one embodiment the glycopeptide antibiotic has the structure shown in FIG. 3.

In one embodiment the glycopeptide antibiotic is vancomycin.

In one embodiment, the concentration of vancomycin present in the antibacterial combination as described herein is below nephrotoxic concentrations for mammalian cells, preferably human cells.

Vancomycin is a currently used antibiotic that belongs to the glycopeptide class and was previously referred to as the drug of last resort for treatment of MRS Staphylococcus aureus infections (Zhou et al., 2015). Other glycopeptide antibiotics are: teicoplanin, telavancin, ramoplanin and decaplanin. Vancomycin was initially discovered in the 1950s but was replaced with new antibiotics discovered concurrently that were more efficient and less toxic. With the emergence of MRSA, vancomycin was brought back to treat MRSA and enterococci and ever since it has been the most successful glycopeptide used to date (Levine, 2006, Yarlagadda et al., 2016). Similarly to other glycopeptides, vancomycin is hydrophilic with a high molecular weight (1.449 Da). In terms of the mode of action, vancomycin has been long known to interfere in bacterial cell wall synthesis until recently additional mechanisms were reported, including induction of a zinc starvation response by chelating Zinc (II) (Zarkan et al., 2016) and enhanced antimicrobial activity via Zinc-mediated polymerization of vancomycin dimers (Zarkan et al., 2017).

The structure of vancomycin (FIG. 3), a glycopeptide antibiotic. This antibiotic was used as a model for demonstrating the synergistic antibacterial effects of certain antibacterial combinations as described herein.

The inventors have surprisingly found that the synergistic action of a triple antibacterial combination comprising a nitrofuran, a bile salt and a glycopeptide antibiotic, preferably a nitrofuran, DOC and vancomycin can be observed in the examples herein that demonstrate that for each of the components in the combination, the minimal inhibitory concentrations (MIC) were decreased below the MICs of those molecules when used in double combinations, effectively lowering the growth-inhibitory concentrations of the individual components when used in the triple combinations. It is important to note here that Gram-negative bacteria are recalcitrant to vancomycin. However, the inventors have found that when used a triple combination, the bactericidal concentration of vancomycin is reduced to a level (15.6 μg/mL) below known nephrotoxic concentrations (30 or 20 μg/mL; depending on the mode and length of treatment) (Elyasi et al., 2012, Mergenhagen and Borton, 2014).

In one embodiment the concentration of vancomycin present the antibacterial combination is from about 1 μg/mL to about 100 μg/mL, preferably from about 2 μg/mL to about 90p/mL, preferably from about 5 μg/mL to about 80 μg/mL, preferably from about 10 μg/mL to about 70 μg/mL, preferably from about 15 μg/mL to about 65 μg/mL, preferably from about 20 μg/mL to about 63 μg/mL, preferably about 20 μg/mL, preferably about 62.5 μg/mL.

In one embodiment the concentration of vancomycin present the antibacterial combination is from 1 μg/mL to 100 μg/mL, preferably from 2 μg/mL to from 90 μg/mL, preferably from 5 μg/mL to from 80 μg/mL, preferably from 10 μg/mL to from 70 μg/mL, preferably from 15 μg/mL to from 65 μg/mL, preferably from 20 μg/mL to from 63 μg/mL, preferably 20 μg/mL, preferably 62.5 μg/mL.

In one embodiment the concentration of vancomycin present the antibacterial combination is less than about 100 μg/ml, preferably less than about 90 μg/mL, preferably less than about 80 μg/mL, preferably less than about 70 μg/mL, preferably less than about 65 μg/mL, preferably less than about 63 μg/mL.

In one embodiment the concentration of vancomycin present the antibacterial combination is less than 100 μg/ml, preferably less than 90 μg/mL, preferably less than 80 μg/mL, preferably less than 70 μg/mL, preferably less than 65 μg/mL, preferably less than 63 μg/mL.

In one embodiment the concentration of vancomycin present the antibacterial combination is less than about 50 μg/ml, preferably less than about 40 μg/mL, preferably less than about 30 μg/mL, preferably less than about 25 μg/mL, preferably less than about 20 μg/mL, preferably less than about 10 μg/mL.

In one embodiment the concentration of vancomycin present the antibacterial combination is less than 50 μg/ml, preferably less than 40 μg/mL, preferably less than 30 μg/mL, preferably less than 25 μg/mL, preferably less than 20 μg/mL, preferably less than 10 μg/mL.

In one embodiment the antibacterial combination comprises about 0.5 μg/mL FZ, about 1250 μg/mL DOC and about 20 μg/mL vancomycin.

In one embodiment the antibacterial combination comprises 0.5 μg/mL FZ, 1250 μg/mL DOC and 20 μg/mL vancomycin.

In one embodiment the antibacterial combination comprises about 8 μg/mL NF, about 1250 μg/mL DOC and about 8 μg/mL vancomycin.

In one embodiment the antibacterial combination comprises 8 μg/mL NF, 1250 μg/mL DOC and 8 μg/mL vancomycin.

In one embodiment the antibacterial combination comprises about 4 μg/mL NFZ, about 625 μg/mL DOC and about 10 μg/mL vancomycin.

In one embodiment the antibacterial combination comprises 4 μg/mL NFZ, 625 μg/mL DOC and 10 μg/mL vancomycin.

In one embodiment the antibacterial combination comprises about 2 μg/mL CM4, about 625 μg/mL DOC and about 10 μg/mL vancomycin.

In one embodiment the antibacterial combination comprises 2 μg/mL CM4, 625 μg/mL DOC and 10 μg/mL vancomycin.

In one embodiment the antibacterial combination inhibits the growth and/or proliferation of at least one Gram-negative bacterial species and/or kills at least one Gram-negative bacterial species. In one embodiment the antibacterial combination is bacteriostatic or bactericidal or both for at least one species of Gram-negative bacteria.

In one embodiment the antibacterial combination is bacteriostatic or bactericidal or both for at least three species of Gram-negative bacteria.

In one embodiment the Gram-negative bacterial species are from the family Enterobacteriaceae.

In one embodiment the at least one Gram-negative species from the family Enterobacteriaceae are chosen from the genera Escherichia, (preferably E. coli); Enterobacter (preferably E. aerogenes and E. cloacae); Salmonella. (preferably S. enteritidis, S. infantis, S. dublin, S. typhimurium, S. paratyphi, S. schottmulleri, or S. choleraesuis); Citrobacter, (preferably Citrobacter gillenii, C. amalonaticus, C. koseri, and C. freundii), Serratia (preferably S. marscences or S. liquifaciens); Shigella (preferably S. sonnei, S. flexneri, S. dysenteriae or S. boydii) and Yersinia spp., preferably Y. enterolitica, Y. pseudotuberculosis or Y. pestis.

In one embodiment the at least one Gram-negative bacterial species is selected from the group consisting of Escherichia coli, Salmonella enterica sv. typhimurium and Citrobacter gillenii.

In one embodiment, contacting is to a plant or part thereof, and the at least one Gram-negative bacterial species is selected from the genera: Vibrio (preferably V. cholerae, V. parahaemolyticus, and V. vulnificus); Neisseria (preferably N. meningitis or N. gonorrhoeae); Acinetobacter (preferably A. baumannii); Bacteroides (preferably B. fragilis); Bordetella (preferably B. pertussis or B. parapertussis); Brucella (preferably B. melitentis, B. abortus or B. suis); Campylobacter (preferably C. jejuni, C. coli or C. fetus); Haemophilus (preferably H. influenzae or H. parainfluenzae); Legionella (preferably L. pneumophila); Pasteurella (preferably P. yersinia or P. multocida); Proteus (preferably P. mirabilis or P. vulgaris).

In one embodiment the species selected correspond to the group consisting of Escherichia coli, Salmonella enterica and Citrobacter gillenii or a strain thereof.

In one embodiment, contacting is to a plant or part thereof, and the at least one Gram-negative bacterial species is selected from the genera: Pseudomonas, (preferably P. tabaci, P. angulata, P. phaseolicola, P. pisi, P. glycinea, P. solanacearum, P. caryophylli, P. cepacia, P. marginalis, P. savastonoi, P. marginata or P. syringae); Xanthomonas (preferably X. phaseoli, X. oryzae, X. runi, X. juglandis, X. campestris or X. vascularum); Erwinia (preferably E. amylovora, E. tracheiphila, E. stewartii or E. carotovora); Agrobacterium (preferably A. tumefaciens, A. rubi or A. rhizogenes).

For the purposes of the present disclosure, an antibacterial combination as described herein is considered “bactericidal” if the addition of an antibacterial combination as described herein to a sample results in the number of colony forming units (cfu) recovered from the sample that is less than about 30%, preferably less than about 25%, less than about 20%, less than about 15%, less than about 10%, or preferably less than about 5% of the cfu recovered from an untreated control sample (i.e., to which the combination has not been added).

In some embodiments the number of colony forming units (cfu) recovered from the treated sample is less than 30%, preferably less than 25%, less than 20%, less than 15%, less than 10%, or preferably less than 5% of the cfu recovered from the untreated control sample.

In some embodiments, an antibacterial combination as described herein reduces the number of cfu that are recovered from a treated sample by less than about 70%, preferably to less than about 60%, to less than about 50%, to less than about 40%, to less than about 30%, to less than about 20%, to less than about 10%, to less than about 5%, to less than about 2%, to less than about 1%, to less than about 0.1%, or preferably to less than about 0.01% of the cfu that are recovered as described herein from an untreated control sample.

In some embodiments an antibacterial combination as described herein reduces the number of cfu that are recovered from a treated sample to less than 70%, preferably to less than 60%, to less than 50%, to less than 40%, to less than 30%, to less than 20%, to less than 10%, to less than 5%, to less than 2%, to less than 1%, to less than 0.1%, or preferably to less than 0.01% of the cfu that are recovered from an untreated control sample.

In one embodiment an antibacterial combination as described herein is in the form of, or is formulated as, a disinfectant.

The formulation of an antibacterial combination as described herein as a disinfectant is believed to be within the skill of those in the art in view of the present disclosure and common general knowledge.

In some embodiments, the combination is formulated as, or is in the form of, a composition comprising an antibacterial combination as described herein and a carrier, diluent or excipient.

In one embodiment the composition consists essentially of the antibacterial combination.

In one embodiment the carrier, diluent or excipient is a buffer. In one embodiment the buffer is a zwitterionic buffer. In one embodiment the zwitterionic buffer is selected from the group consisting of MES, MOPS, HEPES and TRIS, preferably MES. In one embodiment the buffer is an inorganic buffer. In one embodiment the inorganic buffer is selected from the group consisting of citrate, acetate, phosphate and cacodylate. Buffers with low concentrations of chloride ions are preferred to prevent precipitation of AgCl. In one embodiment the buffer maintains the composition in a pH range of about 6 to about 8 or of about 6 to 8 or of 6 to about 8 or of 6 to 8, preferably about 6.5 to about 7.5 or about 6.5 to 7.5 or 6.5 to about 7.5 or 6.5 to 7.5, preferably about pH 6.5, 7 or 7.5, preferably at pH 6.5±0.2, 7±0.2 or 7.5±0.2, preferably at pH 6.5, 7 or 7.5, preferably at pH 6.5.

In one embodiment the composition is a pharmaceutical composition, wherein the carrier, diluent or excipient is a pharmaceutically acceptable carrier, diluent or excipient.

In one embodiment the pharmaceutical composition consists essentially of the antibacterial combination.

In one embodiment the pharmaceutical composition comprises an effective amount of the antibacterial combination, preferably a therapeutically effective amount of the antibacterial combination. In one embodiment the pharmaceutical composition consists essentially of an effective amount of the antibacterial combination, preferably a therapeutically effective amount of the antibacterial combination.

In one embodiment the effective amount is an amount of the antibacterial combination that, when contacted to a Gram-negative bacterial species as described herein, kills at least 50%, preferably at least 60%, at least 70%, at least 80%, at least 90%, at least 95%, at least 98%, at least 99%, at least 99.9%, at least 99.99%, at least 99.995%, or preferably at least 99.999% of the at least one Gram-negative bacterial species as described herein for any aspect of the invention. In this embodiment, the at least one Gram-negative bacterial species comprises a starting population of cells (i.e., the number of cells before treatment begins) of at least 1.0×10⁵ cells.

In one embodiment killing of at least 50%, at least 60%, at least 70%, at least 80%, at least 90%, at least 95%, at least 98%, at least 99%, at least 99.9%, at least 99.99%, at least 99.995%, or preferably at least 99.999% of the Gram-negative bacterial species occurs in less than about 48 hours, preferably less than about 24 hours, preferably less than about 12 hours, preferably less than about 6 hours, preferably less than about 4 hours after the Gram-negative bacterial species is contacted. Preferably killing occurs in less than 4 hours.

In one embodiment killing occurs in less than about 3 hours, preferably less than about 2 hours, preferably less than about 1 hour. In one embodiment killing occurs in less than 3 hours, preferably less than 2 hours, preferably less than 1 hour.

In one embodiment the composition is a cosmetic composition comprising the composition and a cosmetically acceptable carrier, diluent or excipient.

In one embodiment the cosmetic composition consists essentially of the antibacterial combination.

In one embodiment the cosmetic composition is a hair or skin care composition.

In one embodiment the composition is in the form of, or is formulated as a solid, liquid, paste, gel, emulsion, cream, ointment, lotion, liniment, solution, suspension, stick, block, pill, lozenge, powder, slurry, mist or vapour.

The formulation of an antibacterial combination as a composition in the form of a solid, liquid, paste, gel, emulsion, cream, ointment, lotion, liniment, solution, suspension, stick, block, pill, lozenge, powder, slurry, mist or vapour for use to inhibit the growth and/or proliferation of at least one Gram-negative bacterial species, or to treat a Gram-negative bacterial infection, disease and/or condition as described herein is believed to be within the skill of those in the art as described herein and in light of common general knowledge.

In some embodiments, a composition as described herein may also contain other additives such as stabilising agents, preservatives, solubilizers, colouring agents, chelating agents, gel forming agents, ointment bases, pH-regulators, anti-oxidants, perfumes and skin protective agents, but not limited thereto. If the composition is in the form of a shampoo or soap, the composition may further comprise foaming agents, pearling agents and/or conditioners.

Typical preservatives that may be used include the parabens, formaldehyde, Kathan CG, Bronidox, Bronopol, p-chlorom-cresol, chlorhexidine, benzalkonium chloride, etc.

In some embodiments, the compositions of the invention are in the form of a shampoo or a soap. In some embodiments the shampoo or soap comprises additional ingredients selected from the group consisting of betaine, sodium lauryl sulphate, nonylphenol, imidazole, sulphosuccinate, re-fattening agents, humectants, conditioners, and combinations thereof. Conventional ingredients may be used in these embodiments.

An important advantage that the invention provides is that in using bile salts to provide surfactant activity to cosmetic formulations such as soaps, detergents and shampoos, the use of unpopular surfactants such lauryl sulphate redundant in these preparations can be reduced.

In one embodiment the composition, pharmaceutical or cosmetic composition comprises acceptable carriers, particularly pharmaceutically acceptable or cosmetically acceptable carriers, proteins, small peptides, salts, excipients, thickeners, diluents, buffers, preservatives, surface active agents, neutral or cationic lipids, lipid complexes, liposomes, penetration enhancers, carrier compounds and/or other carriers in addition to the antibacterial combination.

Such compositions and formulations can be used as described herein.

An acceptable carrier, particularly a pharmaceutically acceptable or cosmetically acceptable carrier may be liquid or solid and is selected as known in the art, in view of a planned manner of use, application and/or administration. In some embodiments, a pharmaceutically or cosmetically acceptable carrier provides for the desired bulk, consistency, or other desirable pharmaceutical or cosmetic property that is to be used or delivered in a particular context as described herein.

In some embodiments a pharmaceutically or cosmetically acceptable carrier may include binding agents (e.g., pregelatinized maize starch, polyvinylpyrrolidone (PVP) or hydroxypropyl methylcellulose, and the like, fillers such as lactose or other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.).

A person skilled in the art will be able to formulate an antibacterial combination as described herein as a composition, particularly a pharmaceutical or cosmetic composition, by determining an appropriate mode of use, application and/or administration of the composition with reference to the literature and as described herein, and then formulating the composition for such mode with reference to the literature and as described herein. By way of non-limiting example, a formulation of the composition as a pharmaceutical composition for topical application would be preferred for inhibiting the growth and/or proliferation of certain Gram-negative bacteria, or for the treatment and prevention of certain Gram-negative bacterial infections, diseases and/or conditions of the skin or mucosa that are caused by and/or associated with at least one Gram-negative bacterial species. In another non-limiting embodiment, a formulation of the composition as a pharmaceutical composition for systemic application would be preferred for the treatment of systemic or localized internal bacterial infections, diseases and/or conditions of the skin or mucosa that are caused by and/or associated with at least one Gram-negative bacterial species.

In one embodiment the antibacterial combination or pharmaceutical composition is formulated for administration, or is in a form for administration, to a subject in need thereof. In one embodiment administration is selected from the group consisting of is topical, intranasal, epidermal, transdermal, oral or parenteral. In one embodiment parenteral administration is selected from the group consisting of direct application, systemic, subcutaneous, intraperitoneal or intramuscular injection, intravenous drip or infusion, inhalation, insufflation or intrathecal or intraventricular administration. In one embodiment administration is by aerosol delivery.

In one embodiment the antibacterial combination or pharmaceutical composition is formulated for, or is in a form for, parenteral administration in any appropriate solution, including sterile aqueous solutions which may also contain buffers, diluents and other suitable additives.

In one embodiment the antibacterial combination or pharmaceutical composition is formulated for, or is in the form of an injection. In one embodiment, injection is into or near the infected area. In one embodiment the infected area is the ear, eye, nose, throat or mouth.

In one embodiment the antibacterial combination or pharmaceutical composition is formulated for, or is in a form for oral administration in powders or granules, aqueous or non-aqueous suspensions or solutions, sprays, capsules, gels, pills, lozenges or tablets. Thickeners, flavoring agents, diluents, emulsifiers, dispersing aids or binders may be desirable.

In one embodiment the antibacterial combination, pharmaceutical composition or cosmetic composition is formulated for, or is in a form for topical, aerosol, or direct administration in transdermal patches, subdermal implants, ointments, lotions, creams, gels, drops, pastes, suppositories, sprays, liquids and powders. In such embodiments, conventional pharmaceutical and cosmetic carriers, aqueous, powder or oily bases, thickeners and the like may be necessary or desirable.

In one embodiment, the direct administration is direct application or local application. In one embodiment direct or local application comprises application of the antibacterial combination in combination with a delivery reagent or additional anti-microbial agent.

In one embodiment the antibacterial combination or pharmaceutical composition is formulated for injection. In one embodiment, injection is into or near the infected area. In one embodiment the infected area is the ear, eye, nose, throat or mouth.

A person skilled in the art will be able to choose the appropriate mode of administration of an antibacterial combination as described herein with reference to the literature and as described herein. By way of non-limiting example, a systemic application would be preferred for the treatment and prevention of certain microbial infections whereas a local application would be preferred for the treatment of others, but not limited thereto.

In some embodiments, an antibacterial combination or pharmaceutical composition as contemplated herein may be formulated according to conventional pharmaceutical practice and may be: Semisolid formulations: Gels, pastes, mixtures. Liquid formulations: Solutions, suspensions, drenches, emulsions. As indicated, an antibacterial combination or pharmaceutical composition as described herein may comprise a compound of formula I and II, or a functional analogue or derivative thereof, and may also comprise a compound of formula III, or a functional analogue or derivative thereof.

Examples of suitable functional derivatives include pharmaceutically acceptable salts, particularly those suitable for use in a cutaneous environment. Examples include pharmaceutically acceptable salts yielding anions which are pharmaceutically acceptable, particularly in a cutaneous environment. Examples include phosphates, sulphates, nitrate, iodide, bromide, chloride, borate as well as anions derived from carboxylic acids including acetate, benzoate, stearate, etc. Other derivatives of the amino function include amides, imides, ureas, and carbamates but not limited thereto.

Particular examples include salts with pharmaceutically acceptable cations, e.g. lithium, sodium, potassium, magnesium, calcium, zinc, aluminium, ferric, ferrous, ammonium and lower (Cl-6)-alkylammonium salts. Esters include lower alkyl esters.

In some embodiments, a composition as described herein can be formulated as, or provided in the form of a topical composition. As will be understood by a person skilled in the art, a number of different types of topical compositions, including but not limited to topical compositions that are pharmaceutical compositions, can be prepared including peroral, parenteral, intravenous, vaginal, or rectal compositions (but not limited thereto) as described herein, and by following the guidelines for topical application, for example, according to conventional formulation practice, see, e.g., “Remington's Pharmaceutical Sciences” and “Encyclopaedia of Pharmaceutical Technology”. Cosmetic, hair care and skin care compositions may also be prepared as topical compositions. It is believed that the preparation of such topical compositions is also within the skill in the art.

In one embodiment the composition is in the form of, or is formulated as a topical composition. In one embodiment the topical composition is also a pharmaceutical or cosmetic composition as described herein. In one embodiment the antibacterial combination or composition is in the form for, or is formulated for, topical administration. In one embodiment topical administration is to an object or part thereof, preferably to a surface of the object, or a part thereof.

In one embodiment topical administration is to an animal or part of an animal, preferably a mammal, preferably a human. In one embodiment topical administration to an animal comprises administration to a wound, a burn, an ulcer, ulcus curis, acne, gonorrhoea (including urethritis, endocervicitis and proctitis), gas gangrene, scarlatina, erysipelas, sycosis barbae, folliculitis, impetigo contagiosa, or impetigo bullosa.

In some embodiments, topical administration is onto or close to an effected area of the body.

In some embodiments, topical administration is onto an exterior part of the body. In one embodiment the exterior part of the body is the hair or skin or a part thereof.

Topical administration may be by simple application of the composition such as by smearing a créme, ointment, lotion or gel comprising the antibacterial combination onto or around an area to be treated, or from which at least one Gram-negative bacterial species is to be inhibited or killed, or using a soap, detergent, disinfectant or shampoo for the same purpose. Alternatively, it may involve the use of an applicator or device suitable for enhancing the establishment of contact between the combination or composition and the substrate to which it is applied such as by the use of occlusive dressings or plasters comprising the composition or by way of a brush to apply a soap or shampoo. By way of non-limiting example, a composition of the invention may be impregnated or distributed onto pads, plasters, strips, gauze, sponge materials or cotton or wool pieces. In some embodiments, topical administration comprises spraying or misting an area to be treated or from which at least one Gram-negative bacterial species is to be inhibited or killed.

In some embodiments, the topical composition comprises or consists essentially of about 0.001-80%, preferably 0.001-80%, by weight (w/w) of an antibacterial combination of the invention based on the total weight of each component of the combination in the composition. By way of non-limiting example, the topical composition comprises a total of about 0.001-40% w/w, preferably 0.001-40% w/w, of the antibacterial combination wherein the composition comprises three components in the following concentrations: about 0.1-20%, preferably 0.1%-20, about 0.5-10%, preferably 0.5-10%, preferably about 1-5%, preferably about 2-5%, preferably 2-5%.

In some embodiments the topical composition is applied from once to 10 times daily. In some embodiments the topical composition is applied at least once, preferably at least twice, at least three times, at least four times, at least five times, at least six times, at least seven times, at least eight times, at least nine times preferably at least ten times daily. The number of applications may be determined by the skilled person based on the disclosure provided herein and common general knowledge, and will include consideration of the extent to which the growth and/or proliferation of bacteria is to be inhibited in the target area and/or the type, severity and localisation of the bacterial infection, disease and/or condition being treated.

For topical pharmaceutical applications, a composition of the invention may be formulated in accordance with conventional pharmaceutical practice with pharmaceutical excipients conventionally used for topical applications. The nature of the vehicle employed in the preparation of any particular composition will depend on the method intended for administration of that composition.

Vehicles other than water can be used in topical compositions and can include solids or liquids such as emollients, solvents, humectants, thickeners and powders. A skilled worker will appreciate that such vehicles may be used as appropriately in other compositions described herein that comprise the antibacterial combination as described herein including pharmaceutical and cosmetic compositions, as well as disinfectant compositions. Examples of each of these types of vehicles, which can be used singly or as mixtures of one or more vehicles, are as follows:

Emollients, such as stearyl alcohol, glyceryl, monoricinoleate, glyceryl monostearate, propane-1,2-diol, butane-1,3-diol, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate;

Solvents, such as water, methylene chloride, isopropanol, castor oil, ethylene glycol monoethyl ether, diethylene glycol monobutyl ether, diethylene glycol monoethyl ether, dimethyl sulfoxide, tetrahydrofuran, vegetable and animal oils, glycerol, ethanol, propanol, propylene glycol, and other glycols or alcohols, fixed oils; humectants or moistening agents, such as glycerin, sorbitol, sodium 2-pyrrolidone-5-carboxylate, soluble collagen, dibutyl phthalate, gelatin;

powders, such as chalk, talc, kaolin, starch and derivatives thereof, gums, colloidal silicon dioxide, sodium polyacrylate, chemically modified magnesium aluminium silicate, hydrated aluminium silicate, carboxyvinyl polymer, sodium carboxymethyl cellulose, ethylene glycol monostearate;

gelling or swelling agents, such as pectin, gelatin and derivatives thereof, cellulose derivatives such as methyl cellulose, carboxymethyl cellulose or oxidised cellulose, cellulose gum, guar gum, acacia gum, karaya gum, tragacanth gum, bentonite, agar, alginates, carbomer, gelatine, bladderwrack, ceratonia, dextran and derivatives thereof, ghatti gum, hectorite, ispaghula husk, xanthan gum;

polymers, such as polylactic acid or polyglycolic acid polymers or copolymers thereof, paraffin, polyethylene, polyethylene oxide, polyethylene glycol, polypropylene glycol, polyvinylpyrrolidone;

surfactants, such as non-ionic surfactants, e.g. glycol and glycerol esters, macrogol ethers and esters, sugar ethers and esters, such as sorbitan esters, ionic surfactants, such as amine soaps, metallic soaps, sulfated fatty alcohols, alkyl ether sulfates, sulfated oils, and ampholytic surfactants and lecitins; buffering agents, such as sodium, potassium, aluminium, magnesium or calcium salts (such as the chloride, carbonate, bicarbonate, citrate, gluconate, lactate, acetate, gluceptate or tartrate).

For topical applications, the pH of a composition of the invention may be about 3 to about 9, preferably about 4 to about 8, preferably about 5, about 6 or about 7. In some embodiments the pH of a composition of the invention is between 3 and 9, preferably between, 4 and 8, between 5 and 8, between 6 and 8, preferably 7. Depending on the intended use of the composition, buffering agents as described herein and as known in the art may be used by the skilled worker to obtain the pH desired.

In some embodiments, a combination or composition as described herein, preferably a cosmetic composition as described herein, is formulated for, or is in the form of a hair care product or skin care lotion.

In some embodiments, a combination or composition as described herein is formulated as a soap, detergent, body wash, shampoo, lotion, ointment, tooth paste or foam spray.

In one embodiment a combination or composition as described herein is formulated as, or is in the form of a coating.

Many diseases arise from a primary infection with any bacterium, but are caused by the secondary spread of the infectious agent from the primary infection site. Accordingly, the inventors believe that an antibacterial combination as described herein will be useful for inhibiting the growth and/or proliferation of bacteria, including prophylactically, and for treating bacterial infections, diseases and/or conditions in a subject in need thereof. The inventors also believe that the antibacterial combination described herein is useful for the manufacture of a medicament for the prophylaxis and/or treatment of bacterial infections, diseases and/or conditions as described herein.

In another aspect the invention relates to a method of inhibiting the growth and/or proliferation of at least one Gram-negative bacterial species and/or of killing at least one Gram-negative bacterial species comprising contacting the Gram-negative bacterial species with a combination or composition of the invention.

In one embodiment contacting comprises contacting an object or part thereof that comprises the at least one Gram-negative bacterial species. In one embodiment contacting comprises contacting a surface in and/or on the object or part thereof.

In one embodiment contacting is for a sufficient time to allow the combination or composition to inhibit the growth and/or proliferation of the at least one Gram-negative bacterial species on and/or in the object.

In one embodiment sufficient time is at least 30 seconds, preferably at least 1 minute, preferably at least 5 min, preferably at least 10 min, preferably at least 20 min, preferably at least 30 min, preferably at least 40 min, preferably at least 1 hour, preferably at least 2 hours, preferably at least 3 hours, preferably at least 5 hours, preferably at least 12 hours.

In one embodiment sufficient time is about 30 seconds, preferably about 1 minute, preferably about 5 min, preferably about 10 min, preferably about 20 min, preferably about 30 min, preferably about 40 min, preferably about 1 hour, preferably about 2 hours, preferably about 3 hours, preferably about 5 hours, preferably about 12 hours.

In one embodiment contacting comprises directly or indirectly applying the combination or composition to the object or part thereof. In one embodiment applying is directly applying. In one embodiment applying is indirectly applying.

In one embodiment applying comprises applying the combination or composition to the object or part thereof at least two times.

In one embodiment applying is applying as a coating or partial coating.

In some embodiments, applying comprises applying the combination or composition at least 1×, or 2×, or 3×, or 4×, or 5×, or 6×, or 7×, or 8×, or 9×, preferably 10×, or more. In some embodiments applying is least 1× per day (1×/d), at least 2×/d, at least 3×/d, at least 4×/day, at least 5×/day, at least 6×/day, at least 7×/day, at least 8×/day, at least 9×/day, at least 10×/day.

In one embodiment the object is an animal or part thereof, or plant or part thereof.

In one embodiment the animal is a mammal.

In one embodiment the mammal is selected from the group consisting of canines, felines, bovines, ovines, cervines, caprines, porcines, lagomorphs, rodents, camelids and hominids.

In one embodiment the mammal is selected from the group consisting of cats, dogs, rats, stoats, ferrets, possums, guinea pigs, mice, hamsters, zebra, elephants, lions, tigers, cheetah, monkeys, apes, macaques, tarsiers, lemurs, giraffes, prairie dogs, meerkats, bears, otters, tapiers, cows, horses, pigs, sheep, goats, deer, minks, hippopotami and humans.

In one embodiment the animal is a bird selected from the group consisting of chickens, ducks, pheasants, pigeons, ostriches, turkeys and geese.

In one embodiment the part of the animal is the hair, skin or hide, preferably human, cow, deer, sheep or horsehair, skin or hide.

In one embodiment the part of the plant is selected from the group consisting of roots, shoots, stalks, stems, trunks, branches, leaves, buds, flowers, and seeds.

In one embodiment contacting is to an animal or part thereof, and the at least one Gram-negative bacterial species is a species of family Enterobacteriaceae.

In one embodiment the species from the family Enterobacteriaceae are chosen from the genera Escherichia, (preferably E. coli); Salmonella. (preferably S. enteritidis, S. infantis, S. dublin, S. typhimurium, S. paratyphi, S. schottmulleri, or S. choleraesuis); Citrobacter, (preferably Citrobacter gillenii, C. amalonaticus, C. koseri, and C. freundii), Serratia (preferably S. marscences or S. liquifaciens); Shigella (preferably S. sonnei, S. flexneri, S. dysenteriae or S. boydii) and Yersinia spp., preferably Y. enterolitica, Y. pseudotuberculosis or Y. pestis.

In one embodiment the species of Enterobacteriaceae is selected from the group consisting of Escherichia coli, Salmonella enterica and Citrobacter gillenii or a strain thereof.

In one embodiment, contacting is to a plant or part thereof, and the at least one Gram-negative bacterial species is selected from the genera: Vibrio (preferably V. cholerae, V. parahaemolyticus, and V. vulnificus); Neisseria (preferably N. meningitis or N. gonorrhoeae); Acinetobacter (preferably A. baumannii); Bacteroides (preferably B. fragilis); Bordetella (preferably B. pertussis or B. parapertussis); Brucella (preferably B. melitentis, B. abortus or B. suis); Campylobacter (preferably C. jejuni, C. coli or C. fetus); Haemophilus (preferably H. influenzae or H. parainfluenzae); Legionella (preferably L. pneumophila); Pasteurella (preferably P. yersinia or P. multocida); Proteus (preferably P. mirabilis or P. vulgaris).

In one embodiment, contacting is to a plant or part thereof, and the at least one Gram-negative bacterial species is selected from the genera: Pseudomonas, (preferably P. tabaci, P. angulata, P. phaseolicola, P. pisi, P. glycinea, P. solanacearum, P. caryophylli, P. cepacia, P. marginalis, P. savastonoi, P. marginata or P. syringae); Xanthomonas (preferably X. phaseoli, X. oryzae, X. runi, X. juglandis, X. campestris or X. vascularum); Erwinia (preferably E. amylovora, E. tracheiphila, E. stewartii or E. carotovora); Agrobacterium (preferably A. tumefaciens, A. rubi or A. rhizogenes).

In some embodiments inhibiting the growth and/or proliferation of the at least one Gram-negative bacterial species comprise inhibiting or reducing a bacterial infection, disease and or condition caused by or associated with a Gram-negative bacterial species. In some embodiments the bacterial infection, disease or condition is a bacterial infection, disease or condition of tobacco, beans, peas, soybeans, lilac, banana, carnation, kiwifruit, tomato, onion, olive, gladiolus, rice, pears, apples, peaches, cherries, apricots, walnut, almond, cashew, crucifers, citrus, sugar cane, curcurbits, corn, potato, chrysenthemum alfalfa, tomato, raspberries, strawberries, blueberries or elm.

In one embodiment, the combination or composition is formulated as a coating, or is in the form of, a coating or a partial coating.

In one embodiment the combination or composition is formulated as, or is in a form of, a disinfectant, a detergent, a wash, a soap or a shampoo.

In one embodiment, the combination or composition is formulated for use in, or is in a form for use in the leather industry. In one embodiment the combination or composition is formulated for use, or is in a form for use at room temperature or above. In one embodiment room temperature is about 20° C., preferably about 22° C., preferably about 25° C. In one embodiment the combination or composition is formulated for use, or is in a form for use during transportation and/or during storage prior to processing. In this manner a composition of the invention is employed to reduce or prevent hide spoilage which leads to increased risk of disease-causing effluent, worker infection, and product loss due to discoloration and/or degradation.

In one embodiment the object is in inanimate article, material or substance, or part thereof. In one embodiment the object is an object on which Gram-negative bacterial species are known or suspected of being present and/or growing.

In one embodiment the object is used in food processing, hygiene, medicine, dentistry or any other industry where contamination by Gram-negative bacterial species poses a health risk and/or is desired to be prevented and/or reduced. In some embodiments the object is selected from the group consisting of medical devices, surgical devices, surgical instruments, surgical implants, stents, catheters, dental devices, dental instruments dental prostheses, dental implants, contact lenses, bandages, wound dressings, and food processing equipment.

Specifically contemplated as embodiments of this aspect of the invention relating to a method of inhibiting the growth and/or proliferation of at least one Gram-negative bacterial species, and/or of killing at least one Gram-negative bacterial species are all of the embodiments set out herein relating to the aspects of the invention that are the antibacterial combination of the invention and the composition of the invention, and particularly including all specified concentrations of nitrofurans, bile salts and antibiotics.

In another aspect the invention relates to the use of an antibacterial combination or composition of the invention for inhibiting the growth and/or proliferation of at least one Gram-negative bacterial species, and/or for killing at least one Gram-negative bacterial species.

Specifically contemplated as embodiments of this aspect of the invention relating to the use of an antibacterial combination or composition of the invention for inhibiting the growth and/or proliferation of at least one Gram-negative bacterial species and/or for killing at least one Gram-negative bacterial species, are all of the embodiments set out herein relating to the aspect the invention that is a method of inhibiting the growth and/or proliferation of at least one Gram-negative bacterial species and/or of killing at least one Gram-negative bacterial species, including all embodiments within this method aspect that are set forth in the aspects of the invention that are the antibacterial combination of the invention and the composition of the invention, and particularly including all specified concentrations of nitrofurans, bile salts and antibiotics.

In another aspect, the invention relates to a method of treating a Gram negative bacterial infection, disease or condition comprising administering a pharmaceutical composition as described herein to a subject in need thereof.

In one embodiment the bacterial infection, disease and/or condition is caused by or is associated with at least one Gram-negative bacterial species.

In one embodiment the at least one Gram-negative bacterial species is a species of Enterobacteriaceae.

In one embodiment the species of Enterobacteriaceae are chosen from the genera Escherichia, (preferably E. coli); Salmonella. (preferably S. enteritidis, S. infantis, S. dublin, S. typhimurium, S. paratyphi, S. schottmulleri, or S. choleraesuis); Citrobacter, (preferably Citrobacter gillenii, C. amalonaticus, C. koseri, and C. freundii), Serratia (preferably S. marscences or S. liquifaciens); Shigella (preferably S. sonnei, S. flexneri, S. dysenteriae or S. boydii) and Yersinia spp., preferably Y. enterolitica, Y. pseudotuberculosis or Y. pestis. Preferably the species of Enterobacteriaceae are species of Escherichia, preferably E. coli, Salmonella, preferably S. enteritidis, S. infantis, S. dublin, S. typhimurium, S. paratyphi, S. schottmulleri, or S. choleraesuis, or Citrobacter, preferably Citrobacter gillenii, C. amalonaticus, C. koseri, or C. freundii).

In one embodiment the bacterial infection is selected from the group consisting of eye infections, nose infections, ear infections, mouth infections, throat infections, lung infections caused by and/or associated with the at least one Gram-negative bacterial species.

In one embodiment the infection, disease or condition is selected from the group consisting of halitosis, sore throat, orbital cellulitis, conjunctivitis, otitis media, sinusitis, pneumonia, diphtheria, pertussis, epiglottitis, nasopharyngitis, bronchitis, tonsillitis, gastritis, typhus, gastroenteritis, pseudomembranous colitis, dysentery, enterocolitis, peritonitis, abscess, pertussis, cholera, pestis, cystitis, pneumonia, meningitis, and Crohn's disease.

In one embodiment, administration is local or systemic administration. In one embodiment, administration is topical, intranasal, epidermal, and transdermal, oral or parenteral. In one embodiment oral administration comprises aerosol delivery. In one embodiment oral delivery comprises application of a liquid, gel, créme, ointment, or slurry. In one embodiment oral delivery comprises delivery of a solid, preferably a powder.

In one embodiment, parenteral administration is selected from the group consisting of direct application, systemic, subcutaneous, intraperitoneal or intramuscular injection, intravenous drip or infusion, inhalation, insufflation or intrathecal or intraventricular administration.

In one embodiment, administration is transient administration. In one embodiment transient administration comprises administration of an antibacterial combination or composition as described herein for a sufficient period of time to provide a treatment or achieve a therapeutic result without the presence of the antibacterial combination or composition being harmful or causing significant deleterious effects to the subject. Administration can be rapid (e.g., by injection), or can occur over a period of time (e.g., by slow infusion or administration of slow release formulations).

Specifically contemplated herein as embodiments of this method aspect of the invention for treating a bacterial infection, disease and/or condition are all of the embodiments set forth herein relating to the other aspects of the invention that are antibacterial combinations, compositions, pharmaceutical compositions, methods and uses as described herein, particularly as relates to the formulation of compositions, disinfectants and medicaments as described herein, and their subsequent use, application and/or administration as described herein, and particularly including all specified concentrations of nitrofurans, bile salts and antibiotics.

A particular and effective dosage regime according to a method of treating a bacterial infection, disease or condition as described herein will be dependent on severity of the infection, disease and/or condition to be treated and on the responsiveness of the treated subject to the course of treatment. An effective treatment may last from several hours to several days to several months, or until an acceptable therapeutic outcome is effected or assured or until an acceptable reduction of the infection is observed.

An optimal dosing schedule (s) may be calculated from drug accumulation as measured in the body of a treated subject. It is believed to be within the skill of persons in the art to be able to easily determine optimum and/or suitable dosages, dosage formulations and dosage regimes. Of course, the optimum dosages may vary depending on the relative potency of the antibacterial combination or composition as described herein, but will be estimable from an EC50s found to be effective in suitable cells in vitro and in an appropriate in vivo animal model. In general, dosage is from 0.001 g to 99 g per kg of body weight, and may be given once or more daily, weekly, monthly or yearly, but not limited thereto.

In another aspect the invention relates an antibacterial combination or pharmaceutical composition of the invention for use in treating a Gram-negative bacterial infection, disease and/or condition.

Specifically contemplated as embodiments of this aspect of the invention relating to the use of an antibacterial combination or pharmaceutical composition of the invention for treating a bacterial infection, disease and/or condition, are all of the embodiments set out herein relating to the aspect the invention that is a method of treating a bacterial infection, disease and/or condition, including all embodiments within the method of treating aspect that are set forth in the aspects of the invention that are the antibacterial combination of the invention and the composition of the invention, and particularly including all specified concentrations of nitrofurans, bile salts and antibiotics.

In another aspect the invention relates to the use of an antibacterial combination as described herein in the manufacture of a medicament for treating a Gram-negative bacterial infection, disease, and/or condition.

In one embodiment the bacterial infection, disease and/or condition is caused by or is associated with at least one Gram-negative bacterial species. Specifically contemplated for this aspect of the invention are the Gram-negative bacterial species that are set out as embodiments within the context of the antibacterial combination, composition and method aspects of the invention.

In one embodiment the medicament comprises an effective amount of the anti-microbial combination. In one embodiment the effective amount is a therapeutically effective amount. In one embodiment the effective amount in the combination comprises an amount of a glycopeptide antibiotic that is below the nephrotoxic level for a mammalian cell. In one embodiment the effective amount is 20 μg/mL of the antibiotic, or less. Preferably the antibiotic is vancomycin. Preferably the effective amount of vancomycin is about 20 μg/mL, preferably about 15 μg/mL, about 10 μg/mL, about 8 μg/mL, about 6 μg/mL, about 4 μg/mL, preferably about 2 μg/mL.

In one embodiment the medicament comprises less than about 100 μg/ml, preferably less than about 90 μg/mL, preferably less than about 80 μg/mL, preferably less than about 70 μg/mL, preferably less than about 65 μg/mL, preferably less than about 63 μg/mL vancomycin.

In one embodiment the medicament comprises less than 100 μg/ml, preferably less than 90 μg/mL, preferably less than 80 μg/mL, preferably less than 70 μg/mL, preferably less than 65 μg/mL, preferably less than 63 μg/mL vancomycin.

In one embodiment the medicament comprises less than about 50 μg/ml, preferably less than about 40 μg/mL, preferably less than about 30 μg/mL, preferably less than about 25 μg/mL, preferably less than about 20 μg/mL vancomycin.

In one embodiment the medicament comprises less than 50 μg/ml, preferably less than 40 μg/mL, preferably less than 30 μg/mL, preferably less than 25 μg/mL, preferably less than 20 μg/mL vancomycin.

In one embodiment the medicament comprises about 0.5 μg/mL FZ, about 1250 μg/mL DOC and about 20 μg/mL vancomycin.

In one embodiment the medicament comprises about 8 μg/mL NF, about 1250 μg/mL DOC and about 8 μg/mL vancomycin.

In one embodiment the medicament comprises at least one additional anti-microbial agent. In one embodiment the at least one additional anti-microbial agent is an antibiotic. In one embodiment the medicament comprises an effective amount of the additional anti-microbial agent. In one embodiment the effective amount of the at least one additional anti-microbial agent is a therapeutically effective amount.

In one embodiment the medicament consists essentially of an effective amount of the antibacterial combination and an additional anti-microbial agent. In one embodiment the effective amount of the additional anti-microbial agent is a therapeutically effective amount.

In one embodiment the medicament is formulated for administration, or is in a form for administration, to a subject in need thereof.

In one embodiment the medicament is in a form for, or is formulated for topical, intranasal, epidermal, transdermal, oral or parenteral administration. In one embodiment parenteral administration is selected from the group consisting of direct application, systemic, subcutaneous, intraperitoneal or intramuscular injection, intravenous drip or infusion, inhalation, insufflation or intrathecal or intraventricular administration.

In one embodiment the medicament is in a form for, or is formulated for, parenteral administration in any appropriate solution, preferably in a sterile aqueous solution which may also contain buffers, diluents and other suitable additives.

In one embodiment the medicament formulated for, or is in a form for oral administration selected from the group consisting of a powder, a granule, an aqueous suspension, an aqueous solution, a non-aqueous suspension, a non-aqueous solution, a gel, a slurry, an ointment, a créme, a spray, a capsule, a pill, a lozenge, and a tablet.

When administered orally, the addition of one or more of the following may be desirable: thickeners, flavoring agents, diluents, emulsifiers, dispersing aids or binders.

In one embodiment the medicament is formulated for, or is in a form for topical or direct administration selected from the group consisting of transdermal patches, subdermal implants, ointments, lotions, creams, gels, drops, pastes, suppositories, sprays, liquids and powders. Conventional carriers, particularly pharmaceutical carriers, aqueous, powder or oily bases, thickeners and the like may be used as required or desired in this embodiment.

In one embodiment, the direct administration is direct application or local application. In one embodiment direct or local application comprises application of the medicament in combination with a delivery reagent or additional anti-microbial agent.

A person skilled in the art will be able to choose the appropriate mode of administration of the medicament with reference to the literature and as described herein. By way of non-limiting example, a systemic application would be preferred for the treatment and prevention of certain Gram-negative bacterial species infections, diseases and/or conditions, whereas a local application would be preferred for the treatment of others, but not limited thereto.

In one embodiment the medicament is for, is formulated for, or is in a form for administration separately, simultaneously or sequentially with an additional anti-microbial agent.

By way of non-limiting example, one additional anti-microbial agent that may be included in the composition of, or for use in the invention, is an antibiotic that is, or is suspected of being effective against a target cell, particularly a target bacterial cell. In one embodiment the target bacterial cell is a Gram-negative bacterial cell.

In one embodiment the medicament comprises an antibacterial combination as described herein and an antibiotic, wherein the medicament is for, is formulated for, or is in a form for separate, simultaneous or sequential administration of the components in the combination to a subject.

In one embodiment the medicament comprises an antibacterial combination as described herein and an antibiotic, wherein the medicament is for, is formulated for, or is in a form for administration to a subject that has shown a non-response or reduced response to treatment with the antibiotic alone. In another aspect the invention relates to the use of an antibacterial combination as described herein and an antibiotic in the manufacture of a medicament for treating a bacterial infection, disease and/or condition, particularly an infection, disease and/or condition caused by and/or associated with at least one Gram-negative bacterial species.

In one embodiment the medicament is formulated for application to an animal or part thereof. In one embodiment the medicament is in a form for application to an animal or part thereof. In one embodiment the medicament is formulated for administration to an animal. In one embodiment the medicament is in a form for administration to an animal.

Specifically contemplated herein for these aspects of the invention that are the use of an antibacterial combination or composition as described herein in the manufacture of a medicament for treating a bacterial infection, disease, and/or condition are all of the embodiments set out herein relating to the aspects the invention that are the method of inhibiting the growth and/or proliferation of at least one Gram-negative bacterial species and/or of killing at least one Gram-negative bacterial species, and the method of treating a bacterial infection, disease or condition, including all embodiments within these method aspects that are set forth in the aspects of the invention that are the antibacterial combination of the invention and the composition of the invention, and particularly including all specified concentrations of nitrofurans, bile salts and antibiotics.

In another aspect the invention relates to the use of an antibacterial combination of the invention to make a cosmetic composition.

Specifically contemplated herein for this aspect of the invention that is the use of an antibacterial combination as described herein to make a cosmetic composition are all of the embodiments set out herein relating to the aspects the invention that are the method of inhibiting the growth and/or proliferation of at least one Gram-negative bacterial species and/or of killing at least one Gram-negative bacterial species, and the method of treating a bacterial infection, disease or condition, including all embodiments within these method aspects that are set forth in the aspects of the invention that are the antibacterial combination of the invention and the composition of the invention and particularly including all specified concentrations of nitrofurans, bile salts and antibiotics.

Various aspects of the invention will now be illustrated in non-limiting ways by reference to the following examples.

### EXAMPLES

### Material and Methods

Bacterial strains used in these examples are described in Table 1. The plasmids used in these examples are described in Table 2. The introduction of the kan^{R} gene deletion mutations into the wild type strain K1508 from the corresponding Keio collection E. coli K12 knock-out strains (Baba et al., 2006) was performed using phage P1 transduction, according to the standard procedure (Madigan et al., 2009). To eliminate potential polar effects on downstream genes in the operon, the FRT-flanked kan^{R} cassette was then removed using FLP-mediated recombination as previously described. The plasmid pCA24N bearing the gene of interest was purified from the corresponding E. coli strain in the ASKA collection containing ORF expression constructs derived from this organism (Kitagawa et al., 2005) using the ChargeSwitch-Pro Plasmid Miniprep Kit (ThermoFisher Scientific). The plasmid DNA was then chemically transformed into specific E. coli strains for further work.

TABLE 1
Bacterial strains

| Name | Genotype or description | Source |
|---|---|---|
| Escherichia coli UPEC | Isolate from a feline urinary tract infection | New Zealand |
| P191 | | Veterinary Pathology |
| | | (NZVP) diagnostic |
| | | labs, Palmerston |
| | | North, New Zealand |
| Escherichia coli 0157 | Human isolate | Dr. Ann Midwinter, |
| isolate ERL034336 | | School of Veterinary |
| | | Sciences, Massey |
| | | University, |
| | | Palmerston North |
| Salmonella enterica sv. | Type strain, S. enterica subsp, enterica, | ATCC ® 43971 ™ |
| Typhimurium, LT2 | serovar Typhimurium | |
| Salmonella enterica | Pig isolate | Dr. Ann Midwinter, |
| SA223a | | School of Veterinary |
| | | Sciences, Massey |
| | | University, |
| | | Palmerston North |
| Citrobacter gillenii | Isolate from a municipal sewage processing | Rakonjac laboratory, |
| | (water purification) plant, Palmerston | Massey University, |
| | North, New Zealand (classified by complete | unpublished. |
| | 16S rRNA sequencing, 99% identity over | |
| | 1403 nt). | |
| Klebsiella pneumoniae. | Isolate from a municipal sewage processing | Rakonjac laboratory, |
| | (water purification) plant, Palmerston | Massey University, |
| | North, New Zealand (classified by complete | unpublished |
| | 16S rRNA sequencing; 99% identity over | |
| | 1403 nt). | |

Escherichia coli K12
laboratory strains:

| | | |
|---|---|---|
| K1508 | MC4100 [F⁻ araD⁻ Δlac U169 relA⁻ thiA rpsL | (Spagnuolo et al., |
| | (Str^{R})] ΔlamB106 | 2010) |
| BW25113 | rrnB3 ΔlacZ4787 hsdR514 Δ(araBAD)567 | (Baba et al., 2006) |
| | Δ(rhaBAD)568 rph-1 | |
| K2403 | K1508, ΔtolC | Le, V. H. V. & |
| | | Rakonjac, J., |
| | | unpublished |
| K2424 | K1508, ΔacrA | Le, V. H. V. & |
| | | Rakonjac, J., |
| | | unpublished |
| K2425 | K1508, ΔacrA, pCA24N-acrA | Le, V. H. V. & |
| | | Rakonjac, J., |
| | | unpublished |
| K2426 | K1508, ΔtolC, pCA24N-tolC | Le, V. H. V. & |
| | | Rakonjac, J., |
| | | unpublished |
| K2433 | K1508, pCA24N-hmp | Le, V. H. V. & |
| | | Rakonjac, J., |
| | | unpublished |
| K2524 | K1508, pUC118 (Amp^{R}). | Le, V. H. V. & |
| | | Rakonjac, J., |
| | | unpublished |

TABLE 2
Plasmids

| Name | Description | Source |
|---|---|---|
| pCP20 | Amp^{R}, Cm^{R}, FLP⁺, 8 cI857⁺, 8 p_{R} Rep^{ts} | (Cherepanov and |
| | For removal of an frt-flanked kan marker from | Wackernagel, 1995) |
| | E. coli K12 strains by FLP-mediated site specific | |
| | recombination | |
| pCA24N-tolC | Cm^{R}; lacI^{q}, pCA24N P_{T5-lac}::tolC Δgfp | (Kitagawa et al., 2005) |
| pCA24N-acrA | Cm^{R}; lacI^{q}, pCA24N P_{T5-lac}::acrA Δgfp | (Kitagawa et al., 2005) |
| pCA24N- hmp | Cm^{R}; lacI^{q}, pCA24N P_{T5-lac}::hmp Δgfp | (Kitagawa et al., 2005) |
| pUC118 | Amp^{R}, f1 ori, P_{lacUV5}, lacZα | Creative Biogene, |
| | | Shirley, NY, USA |

E. coli was grown in 2×YT medium (BD Difco) at 37° C. with shaking at 200 rpm. For preparation of exponential phase cells, fresh overnight culture was 100-fold diluted and incubated to reach the OD₆₀₀ₙₘ of about 0.1-0.2. This cell suspension was then diluted to the desirable concentration depending on specific purposes. Antibiotics used in this study include ampicillin (100 μg/ml, GoldBio), kanamycin (50 μg/ml, GoldBio), tetracycline (10 μg/ml, Boehringer Mannheim), chloramphenicol (30 μg/ml, Sigma), streptomycin (25 μg/ml, Sigma), sodium deoxycholate (New Zealand Pharmaceuticals Ltd.), nitrofurantoin (GoldBio), furazolidone (FZ) (GoldBio) and vancomycin (GoldBio).

### Checkerboard and Dose-Response Growth Inhibitory Assays

The checkerboard and dose-response bacterial growth inhibition assays were performed in Corning 384-well microtiter plates. The concentrations of nitrofurans and sodium deoxycholate (DOC) or vancomycin (Van) were prepared by 2-fold serial dilutions as required for a particular assay. For analysis of triple antibiotic concentrations multiple checkerboard assays of FZ against Van were carried out, each checkerboard assay including DOC at a specific concentration over the tested range.

In the growth inhibition checkerboard and dose-response assays each well contained a starting inoculum of approximately 10⁶ CFU/ml, 2% DMSO and a predefined concentration of each drug in the total volume of 50 μl. The wells containing no drugs and 10 μg/ml of tetracycline were used as negative and positive controls, respectively. After dispensing the reagents, the plate was pulse-centrifuged at 1000×g to eliminate any bubbles. The plate was then incubated at 30° C. and the OD₆₀₀ₙₘ of the sample was monitored for every 1 h within 24 h using Multiskan™ GO Microplate Spectrophotometer (Thermo Scientific). Each combination was performed in triplicate.

The growth inhibition with the cut-off value of 90% at the time point 24 h was used to define the minimum inhibitory concentration (MIC) of the drug used alone or in combination. The Fractional Inhibitory Concentration index (FICi) for any two drugs (e.g. A and B) were calculated as follows: FICi=FIC_{A}+FIC_{B}, where the fractional inhibitory concentration (FIC) of each drug was calculated as MIC_{Acomb}/MIC_{Aalone} (i.e. the ratio of 90% growth inhibition when applied in combination vs. alone.)

The interaction between two drugs was interpreted as synergistic if FICi was ≤0.5, indifferent if it was >0.5 and ≤4, and antagonistic if it was >4. The interaction between two antibacterial agents was plotted using isobologram graphs where >90% inhibition concentrations or FICs of one drug was plotted against those of the other drug (Doern, 2014).

### Time Kill Assay

Exponential phase culture at about 10⁶ CFU/ml was prepared in the final volume of 10 ml containing DMSO 2%. Compounds were used alone and in combination. Furazolidone was used at 1.25 μg/ml and DOC at 2500 μg/ml; Van was at 62.5 μg/ml when used in combination and 500 μg/ml when used alone. The treatment containing no drugs was used as negative control. Tetracycline (Tet) is an antibiotic that inhibits growth, but does not kill bacteria and was used as a bacteriostatic control. The samples were incubated at 30° C. with shaking at 200 rpm. At the time points of 0 h, 2 h, 4 h, 6 h, 8 h and 24 h, 500 μl were taken from each treatment and centrifuged at 10000×g for 15 min before being re-suspended in 100 μl of maximum recovery diluent (MRD, 0.1% peptone, 0.85% NaCl). Serial dilutions 10 μl were each plated on 2×YT agar, followed by overnight incubation at 37° C. to determine the cell count. Each treatment was performed in triplicate. The antibacterial interaction was interpreted as synergistic if the combinatorial treatment caused a killing efficiency ≥100-fold higher than the most active agent (Doern, 2014).

### Assay of Deoxycholate-Furazolidone Combination Antibacterial Efficacy on Meat

A beef rump steak package was purchased from a local supermarket (Palmerston North, New Zealand). The beef surface was sterilized with 70% ethanol before being removed using a sterile scalpel. The inner portion of the meat which was considered free of microorganisms was cut into slices with the surface area of one side of about 4 cm². Each slice was transferred to a Petri dish, and labeled as pre-inoculation, pre-treatment, vehicle-treated and test samples. Each type of treatment was performed in triplicate. For the pre-inoculation samples, a cotton swab (pre-moistened in the maximal recovery diluent (MRD) was passed over the specified 4 cm² area, 5 times horizontally and 5 times vertically. The swab was then vigorously suspended into 0.5 ml of MRD to release any collected bacteria. The resulting cell suspension was then 10-fold serially diluted and 25 μl of each dilution was spread onto 2×YT agar supplemented with 25 μg/ml Streptomycin—a marker present in K1508 genome that distinguishes this strain from potential meat-colonizing bacteria. The plates were then incubated overnight at 37° C. for colony enumeration.

For inoculation, 400 μl of exponential phase E. coli suspension (strain K1508) at the concentration of about 10⁷ CFU/ml were placed onto 4 cm² of each slice of meat. The samples were incubated at room temperature for 20 min for cell attachment. The inoculated samples were then treated as the same way for pre-inoculation samples to determine the number of E. coli cells having attached to meat surface.

Following the inoculation, PBS solution (pH 7.4) was sprayed onto the vehicle-treated samples while the DOC/FZ solution (prepared in PBS pH 7.4) at the concentrations of 2,500 μg/ml and 0.32 μg/ml, respectively, was sprayed onto the test samples. The application was completed within 5 seconds. The samples were then incubated at 30° C. for 2 hours. After that, the cell count was determined as previously described.

In an attempt to simulate the slaughterhouse conditions, the samples after PBS or antibacterial treatment were left at room temperature for 10 min instead of 2 h at 30° C. in another experiment. The subsequent steps leading to bacterial enumeration were performed in the same way as described above.

### Assay of Deoxycholate-Furazolidone Combination Antibacterial Efficacy on Hides

Cow hide was stored at −80° C. before use. The hide was thawed at room temperature before being cut into slices of the size of 2 cm×0.5 cm×0.5 cm (length×width×thickness) and transferred to a 10 ml Falcon tube. 5 ml of MRD was then added into the tube and vigorously vortexed for 30 s. After that, the liquid was discarded. Following that, the sample was inoculated with 5 ml of 10⁶ CFU/ml E. coli K1508 at room temperature for 10 min. The liquid was then disposed of and the hide sample was transferred into a new tube, where it was treated with 10 ml of DOC (2,500 μg/ml) FZ (0.32 μg/ml). The samples treated with sterile water were included as vehicle treated controls. The samples were then incubated at 30° C. for 6 h. After that, the liquid was discarded and cell enumeration was performed. This was completed by vortexing the hide sample in 5 ml MRD vigorously for 30 s, followed by preparing 10-fold serial dilutions and plating 10 μl of each dilution on 2×YT agar plus Streptomycin 25 μg/ml selecting for K1508 strain (which carries a chromosomal mutation in the rpsL gene that results in resistance to Streptomycin; see Table 1). Determination of cell number was also performed at the stages before inoculation and before treatment. Each treatment was performed in triplicate.

### Example 1

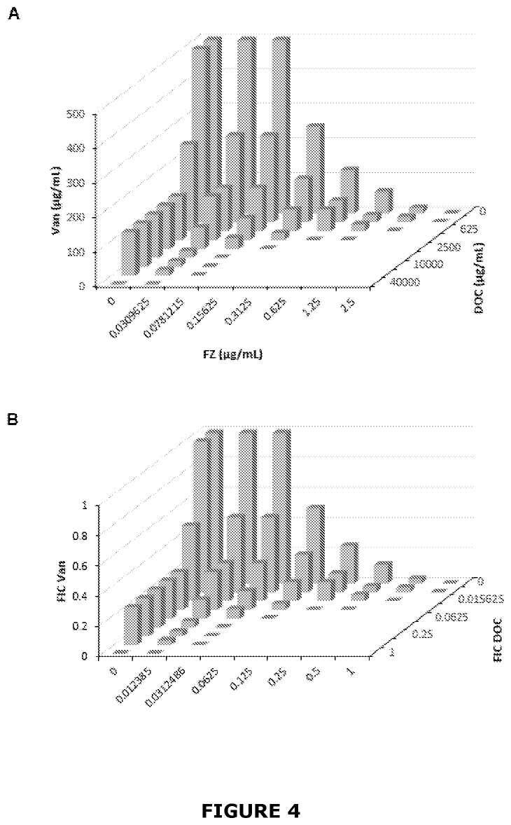

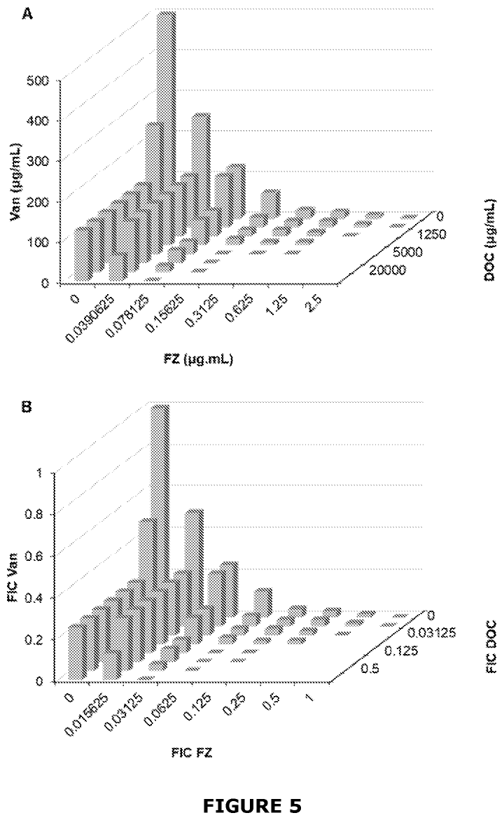

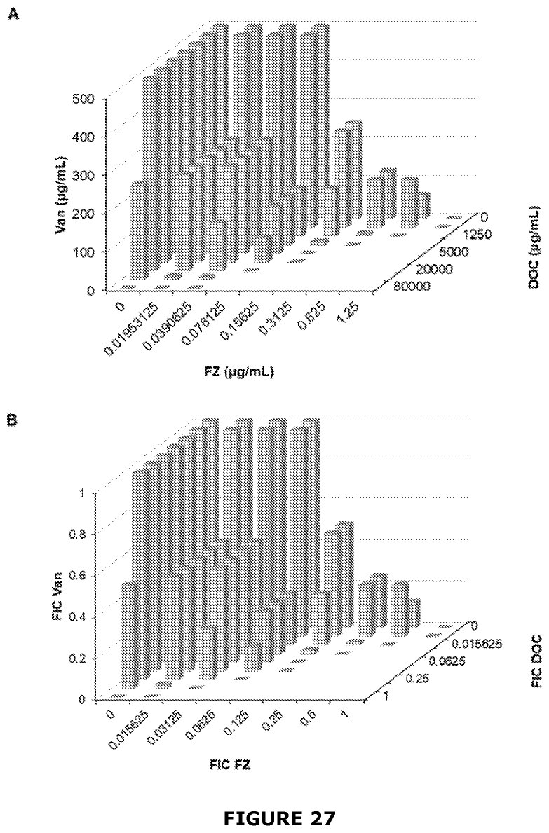

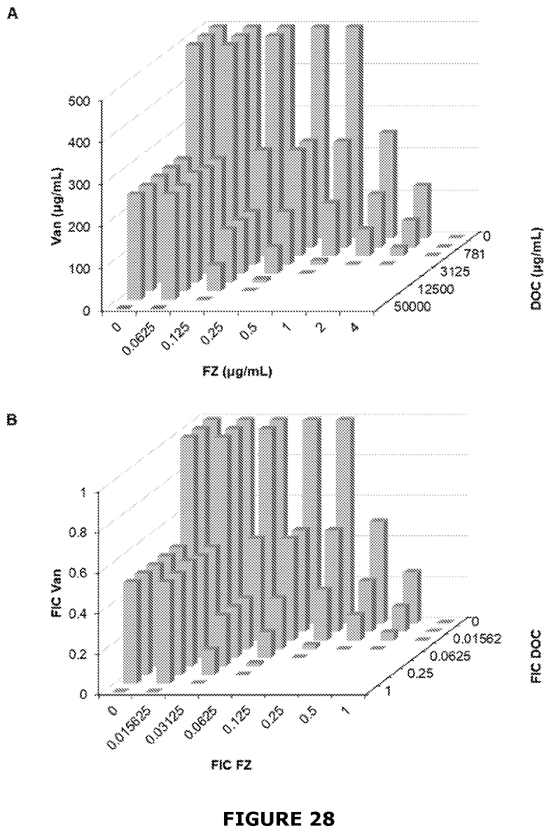

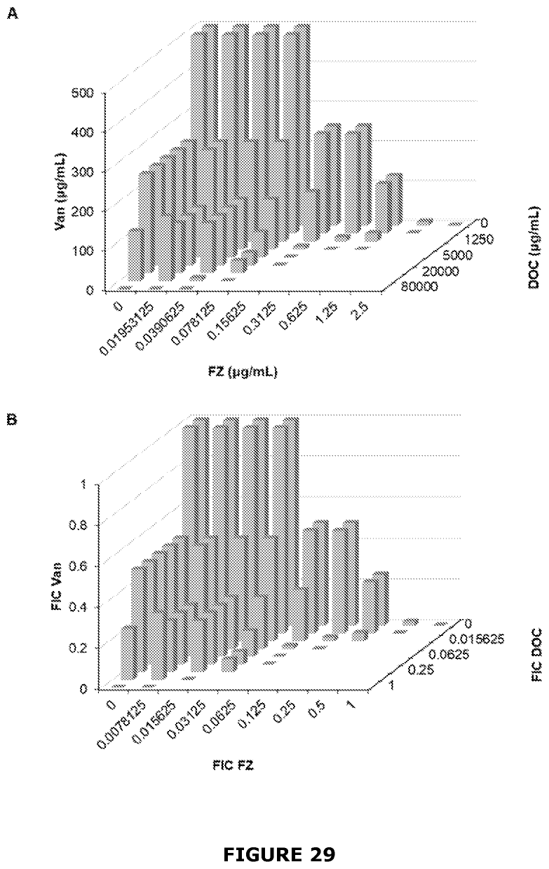

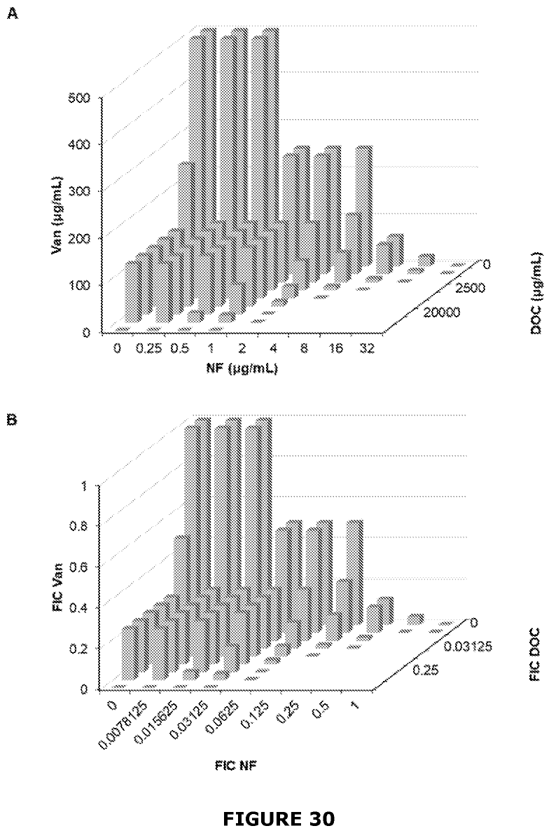

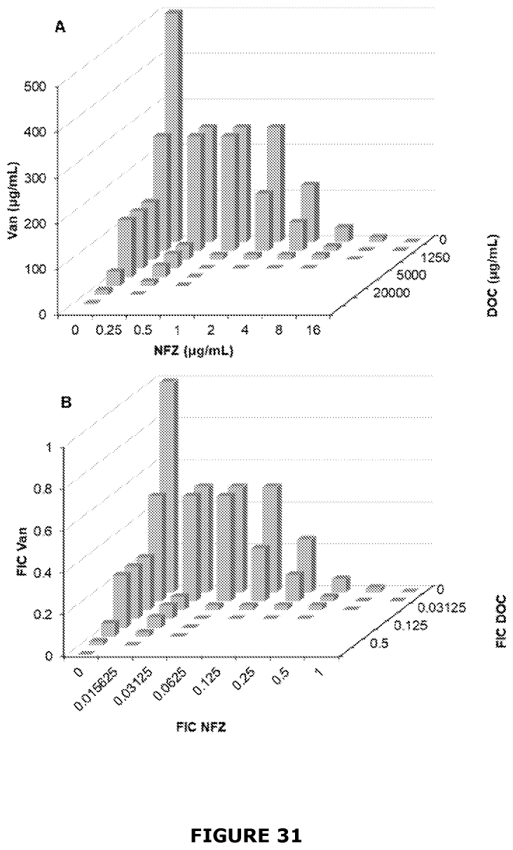

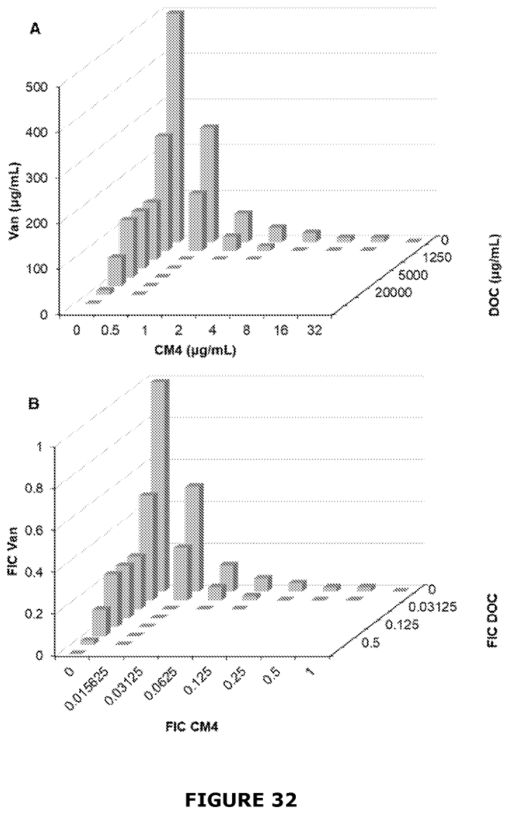

In a first example we demonstrated the synergistic three-way interaction in growth inhibition assay of furazolidone (FZ), deoxycholic acid (DOC) and vancomycin (Van) against Enterobacteria E. coli strains K12 and O157, (FIGS. 4 and 5, respectively), Salmonella enterica sv. typhimurium LT2 (FIG. 27), Citrobacter gillenii (FIG. 28) and E. coli UTI isolate (FIG. 29). The latter E. coli UTI isolate was also subjected to the three-way growth inhibition assay where furazolidone (FZ) was replaced by nitrofurantoin (NF; FIG. 30). Three-way growth inhibition assays of E. coli K12, where FZ was replaced by NFZ or CM4, were also performed (FIGS. 31 and 32, respectively).

A growth inhibition assay was used where a series of concentrations of each antibacterial was applied combined with several concentrations of other two antibacterials (7 concentrations of Van, 8 of FZ and 9 of DOC). FZ concentrations ranged from 0 to 2.5 μg/ml, DOC from 0 to 40 mg/ml and Van from 0 to 500 μg/ml. Starting from the highest concentrations, two-fold dilution series were assayed. E. coli cultures were seeded with ˜10⁶ cells/mL and incubated overnight. Optical densities of each combination of concentrations were measured to assess growth and the inhibition relative to the no-antibiotic control were calculated. The combinations of lowest concentrations that resulted in >90% growth inhibition were plotted on a 3-dimensional graph (FIGS. 4A-5A and 27A-29A). The synergistic interaction has been defined by the Fractional Inhibitory Concentration index (FICi); see the Definitions and Material and Methods sections. FICi represents the sum of the individual Fractional Inhibitory Concentrations (FIC). An FIC in turn is equal to the ratio of concentration that inhibits growth (>90%) in combination vs. alone. For pairwise combinations, the FICi below 0.5 (below 0.25 for each antibiotic) is considered synergistic; by extension for triple combination the FICi should be <0.75 (again below 0.25 for each antibiotic). The FICi for the triple combination on E. coli K12 and O157, respectively, can be calculated from FIGS. 4B and 5B that plotted the FIC values was 0.1625 [0.03125 (Van)+0.0625 (FZ)+0.0625 (DOC)], and 0.125 [0.03125 (Van)+0.0625 (FZ)+0.03125 (DOC)], therefore well below the threshold. Triple combination FICs for S. typhimurium, Citrobacter gillenii and E. coli UTI isolates were also below the threshold (0.172-0.176; FIG. 27-29). Furthermore, FIC values of the triple combinations where FZ was replaced by NF, NFZ and CM4 were 0.172, 0.082 and 0.100, respectively (FIG. 30-32). Notably, the concentration of Van required to inhibit E. coli K12 and O157 was lowered from 500 μg/ml to 20 μg/ml and from 250 μg/ml to 16 μg/ml, respectively, at the combination of concentrations at the lowest FICi point. At several points in the graph Van concentration was lowered to 16 μg/ml and 10 μg/ml, concentrations below the nephrotoxic threshold.

Generally speaking, the concentration of vancomycin required to inhibit various Gram-negative bacteria as disclosed herein were lowered from about 500 μg/mL to about 10 μg/mL, with some variation observed between strains. In some cases the concentration of vancomycin was less than 10 μg/mL.

The inventors believe that the data presented in example 1 allows a skilled person in the art to form a sound scientific prediction that nitrofuran drugs, and in particular the furazolidone tested as described herein, when combined with DOC, and Van, will demonstrate synergistic growth-inhibitory effects on Enterobacteria E. coli (laboratory and pathogenic strains), S. typhimurium, and C. gillenii.

### Example 2

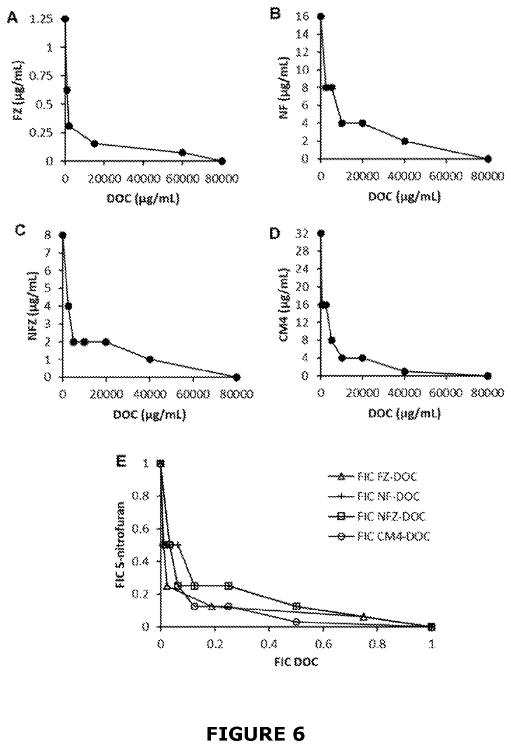

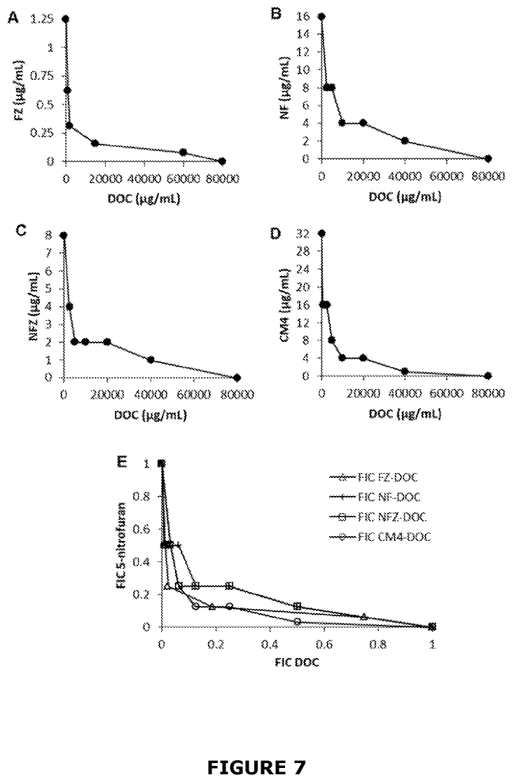

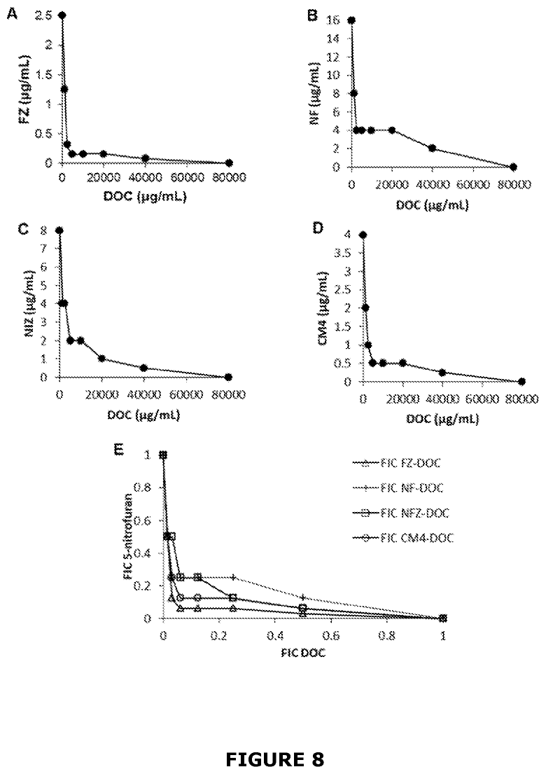

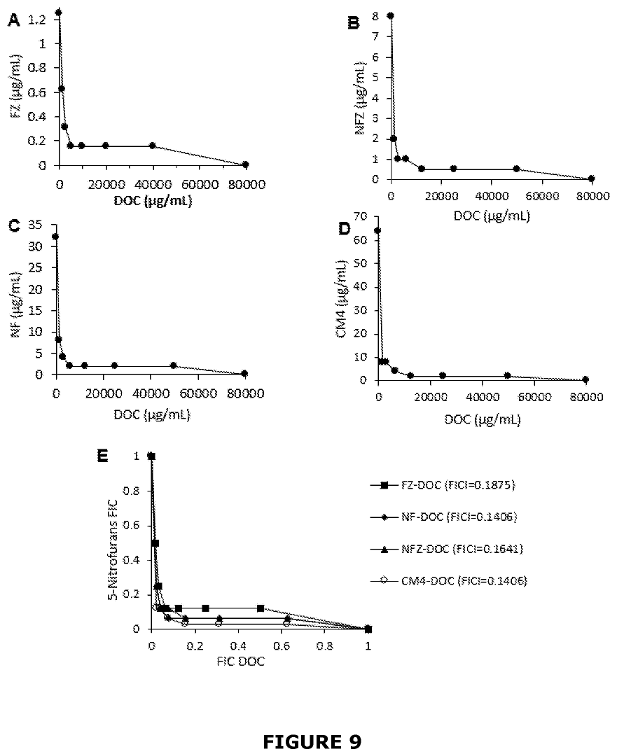

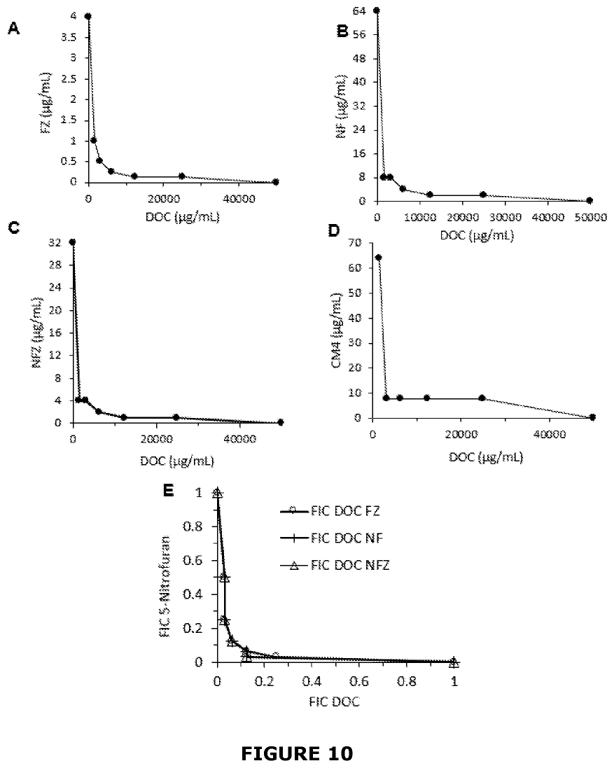

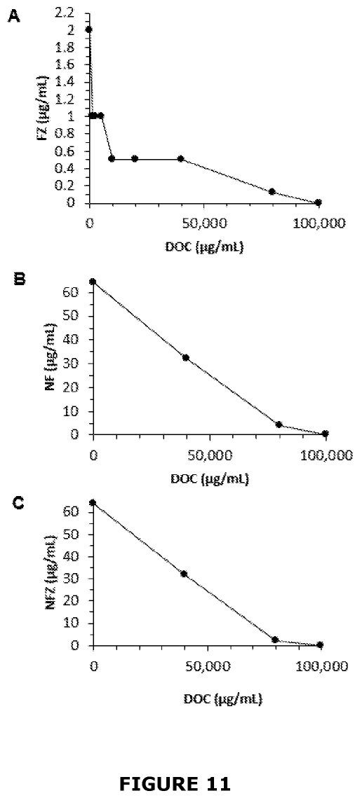

Triple synergy is based on the pairwise synergies of the three antibacterials, FZ (a nitrofuran), DOC and Van. To start understanding the triple interaction, a series of experiments analysing pairwise combinations were carried out. In the first series (FIG. 6-10), synergy in growth inhibition of four enterobacterial species, Escherichia coli (laboratory strains, an O157 isolate and a UTI isolate) S. typhimurium (Laboratory strain LT2), Citrobacter gillenii (a wastewater purification plant isolate), has been demonstrated between DOC and four nitrofuran antibacterials: furazolidone, (FZ; FIG. 1A), nitrofurantoin (NF; FIG. 1B) and nitrofurazone (NFZ; FIG. 1C) and CM4 (FIG. 1D) which has no CAS number assigned and is not used as a drug (it is produced at a small scale and included in small-molecule libraries produced by Enamine, France). In contrast to bacteria from the genera Salmonella, Escherichia and Citrobacter (FIG. 6-10), a Klebsiella pneumoniae strain (a wastewater purification plant isolate) showed synergy for FZ, but not for NF and NFZ (FIG. 11). In addition, CM4 did not inhibit growth of this Klebisiella strain in the range of concentrations used in the experiment (up to 256 μg/ml). In conclusion, Salmonella, Escherichia and Citrobacter are susceptible to synergistic action of all tested nitrofurans with DOC. In contrast, Klebsiella is susceptible to synergistic action of FZ (but not NF and NFZ) with DOC and is resistant to nitrofuran CM4.

The inventors believe that the data presented in example 2 allows a skilled person in the art to form a sound scientific prediction that nitrofuran drugs, and in particular the four nitrofurans tested as described herein, when combined with DOC, will demonstrate synergistic growth-inhibitory effects on Enterobacteria, including E. coli, Salmonella enterica sv. typhimurium (S. typhimurium) and Citrobacter gillenii.

### Example 3

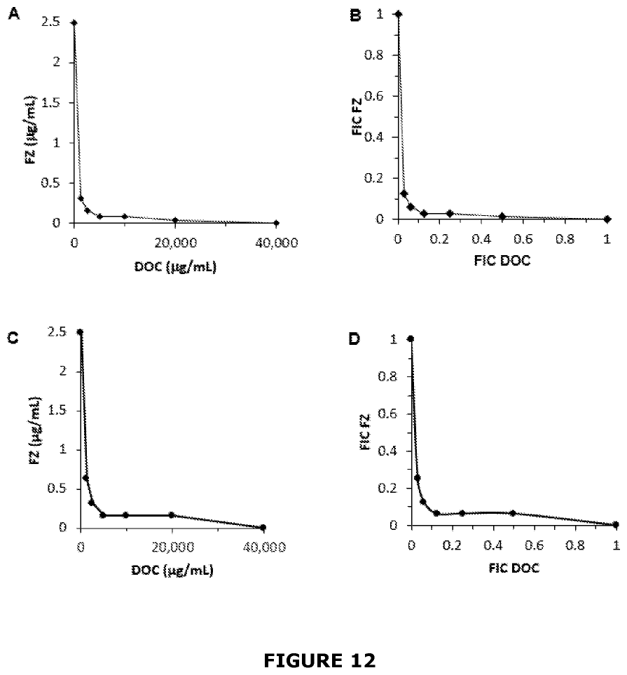

Examples 1 and 2 provide evidence of enterobacterial growth inhibition by synergistic action of a triple combination containing a nitrofuran, DOC and Van, or double combination of a nitrofuran and DOC. Given that current antibacterial resistance threat comes chiefly from enterobacteria that are resistant to the broad family of β-lactam antibiotics, it was of interest to analyse the nitrofuran-DOC synergy in a β-lactam antibiotic-resistant E. coli (FIG. 12). The tested strain (E. coli K12 K2524 (ampicillin-resistant; produces β-lactamase). This strain is also streptomycin-resistant due to a target mutation (rpsL; see Table 1 for genotypes and Table 2 for plasmid details).

### Example 4

Examples 1, 2 and 3 provide evidence of growth inhibition by triple and double combinations. In many applications, it is more important to kill bacteria to resolve an infection rather than solely to stop them from dividing.

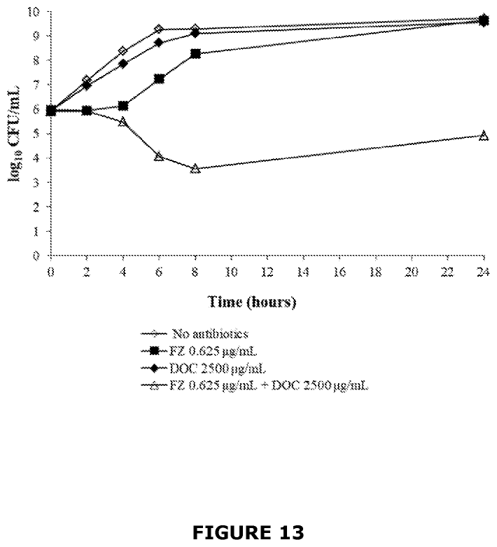

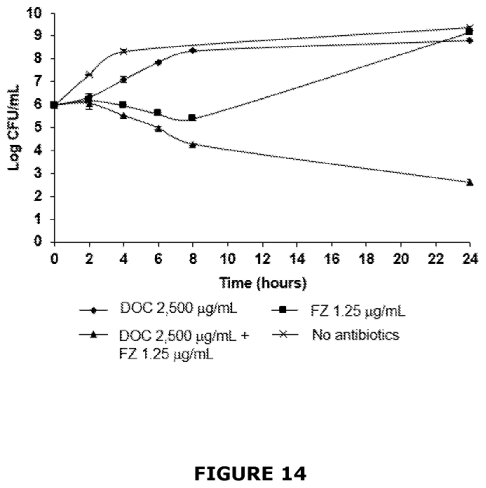

In order to determine whether the synergistic combination is bactericidal, it was examined in another example whether DOC and FZ act synergistically in killing bacteria using a time-kill assay. The S. typhimurium LT2 at a starting inoculum of about 10⁶ CFU/mL was exposed to DOC 2,500 μg/ml alone, FZ 0.625 μg/ml alone and the combination of the two drugs at these concentrations. The titer of viable bacteria was monitored over a time-course of 24 h at 30° C. (FIG. 13). Similar experiment was carried out with E. coli K12 laboratory strain K1508 (FIG. 14), except that the FZ concentration was 1.25 μg/ml. Each concentration was sub-inhibitory when the DOC and FZ were used alone in growth inhibition assay of E. coli K12 K1508, but were inhibitory in combination.

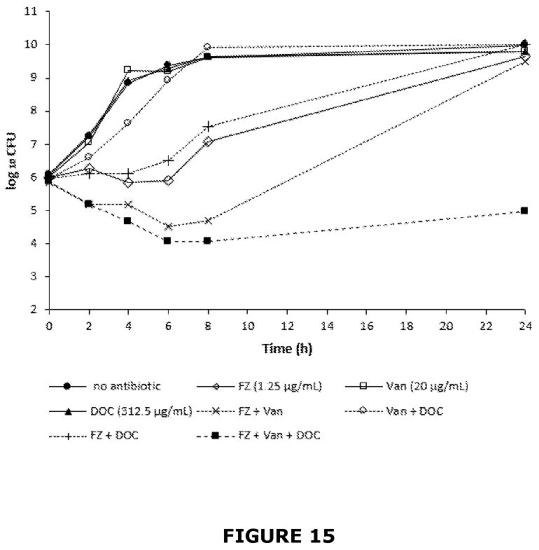

The time-kill experiment of E. coli K12 K1508 was expanded to include the triple combination of antibacterials, where FZ concentration was 1.25 μg/ml, DOC 312.5 μg/ml and vancomycin 20 μg/ml (FIG. 15). Cultures containing single antibiotics and double combinations were not lethal, however the triple combination was effective in killing E. coli.

After 24 h, the total cell count in the sample treated with the triple DOC, FZ and Van combination (FIG. 15) double DOC and FZ mixture was five to six orders of magnitude lower than that in the sample treated with double combinations (FIG. 15) or any of the antibacterials alone. The antibacterial interaction is interpreted as synergistic in the time-kill assay if the combinatorial treatment caused a killing efficiency 100-fold higher than the most active agent (Doern, 2014). Therefore, the synergy between DOC and FZ was confirmed in killing E. coli K-12 strain K1508 or S. typhimurium LT2. Of another note is that double combinations (FIGS. 13 and 14) and triple combination (FIG. 15) decreased the viable titres by two orders of magnitude within 8 hours of incubation and can therefore be considered bactericidal.

### Example 5

True synergy is defined as action of one antibacterial on a target in a bacterium to enhance action or retention of the other antibacterial (in contrast to simple additive effect of inhibiting two parallel vital processes). This implies that candidate genes could be identified that encode proteins executing the processes that increase resistance to one drug, but are hypothesized to be inactivated by other drug.

A hypothesis was proposed that the nitrofurans have inhibitory activity on the AcrAB-TolC pump that is the chief pathway for expulsion of DOC from E. coli cells (Nishino and Yamaguchi, 2001, Paul et al., 2014). If this scenario was true, disruption of the function of efflux pumps by deletion of the genes encoding the pump components would make this activity of FZ redundant, thus reducing the fractional inhibitory concentration index (FICi) in the corresponding deletion mutant strains.

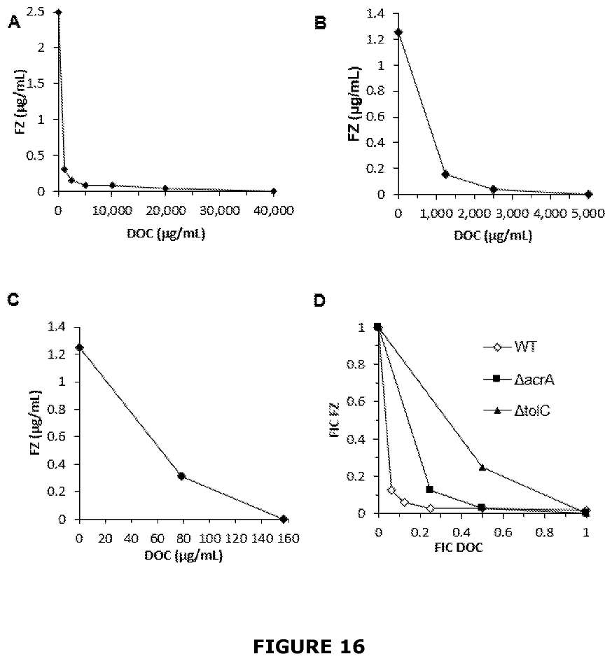

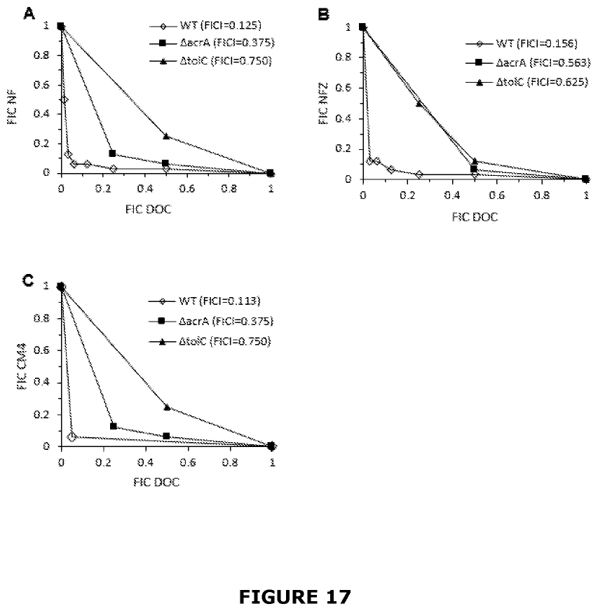

To validate this hypothesis, DOC-FZ synergy was assayed on the ΔtolC and the ΔacrA mutants, which lack the outer membrane and the periplasmic component, respectively, of the AcrAB-TolC efflux pump system, using the checkerboard growth inhibition assay. Deletion of to/C gene caused a shift from a synergistic (FICI=0.125; parental wild-type strain) to an indifferent interaction (FICI=0.75; FIG. 16A; C; ΔtolC). Similarly, the ΔacrA mutant also exhibited a 3-fold increase of the FICi relative to the wild-type (FIG. 16B). The DOC synergy with each of the three other nitrofurans, NF, NFZ and CM4, was converted to an indifferent interaction on deletion of to/C or acrA genes (FIG. 17).

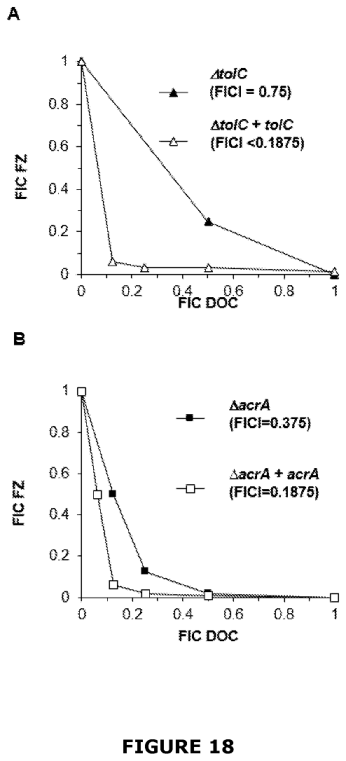

To confirm that these observations were conferred by the removal TolC or AcrA proteins and inactivation of the AcrAB-TolC efflux pump, rather than indirect effect on other genes, we performed complementation assays for the corresponding deletion mutations (FIG. 18). In these experiments, plasmids expressing TolC and AcrA were introduced into the deletion strains, ΔtolC and ΔacrA, respectively. These are multicopy plasmids were derived from an expression vector (pCA24N). Transcription of inserted genes (in this case to/C or acrA) in this vector is controlled by an inducible promoter which, however, has a low level of constitutive expression even in the absence of inducer (Kitagawa et al., 2005). We have performed complementation experiments in the absence of inducer since overexpression of membrane proteins is known to inhibit cell growth, thus evaluation was based on the basal expression of TolC and AcrA from the high copy number pCA24N plasmid. As expected, we demonstrated that strong synergy between DOC and FZ was restored in these complemented strains (FIG. 18A; B). These findings collectively support the model that the TolC-dependent efflux pumps, in particular AcrAB, are the interacting point for the synergy between DOC and FZ.

TolC serves as the outer membrane component of multiple efflux pumps in E. coli whereas AcrA is only a component of the single (TolC-ΔcrAB) pump. We found that the synergy of FZ, NF and CM4 was more severely affected by the ΔtolC than ΔacrA mutation suggesting involvement of additional inner membrane transporters with which TolC interacts in the synergy of these three nitrofurans with DOC. In contrast, the NFZ-DOC synergy was affected to the same extent by ΔtolC than ΔacrA, suggesting the sole involvement of the AcrA-dependent pump.

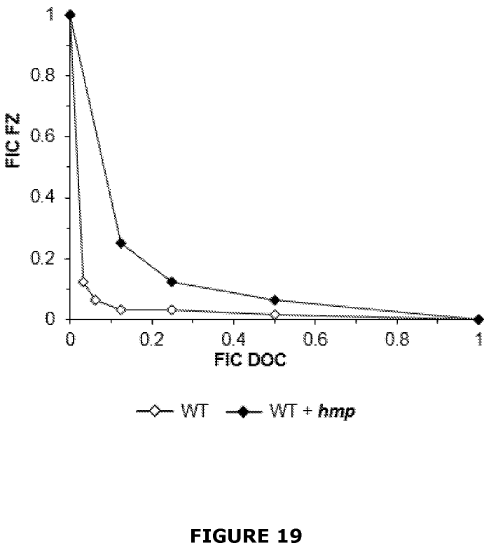

To understand how nitrofurans inhibit the TolC-AcrAB pump, we hypothesized that inhibition of efflux pumps during DOC-FZ interaction was is via Nitric Oxide (NO) generation from FZ. Nitric oxide has been reported to inhibit bacterial electron transport chain and thus interfere with the maintenance of proton motive force across the membrane (Vumma et al., 2016). Given that proton motive force is used as energy supply by efflux pumps directly, or indirectly via ATP synthesis, if FZ acts on efflux pumps via production of NO, proteins involved in NO detoxification in E. coli should decrease the pump inhibition by FZ and in turn the synergy with DOC. To verify the proposed hypothesis, the interaction between DOC and FZ in the E. coli strain with increased expression of protein Hmp (an E. coli nitric oxide dioxygenase, which serves to remove NO) was inspected. The rationale for this is that overexpression of Hmp protein would increase detoxification of NO by conversion into benign No₃⁻ ions, thus relieving the effect exerted by NO (Forrester and Foster, 2012) and decreasing the synergy. In confirmation of this hypothesis, the induction of the hmp expression by 1 mM IPTG was found to increase the FICi (and decrease the synergy) between DOC and FZ by more than two-fold (FIG. 19). This finding supports the hypothesis that NO generated during FZ metabolism in E. coli participates in the inhibition of electron transport chain. Due to a relatively small decrease in synergy NO seems to be only one of the factors secondary to FZ activity that mediate the DOC synergy.

### Example 6

The Efficacy of DOC-FZ Treatment on E. coli-Inoculated Meat

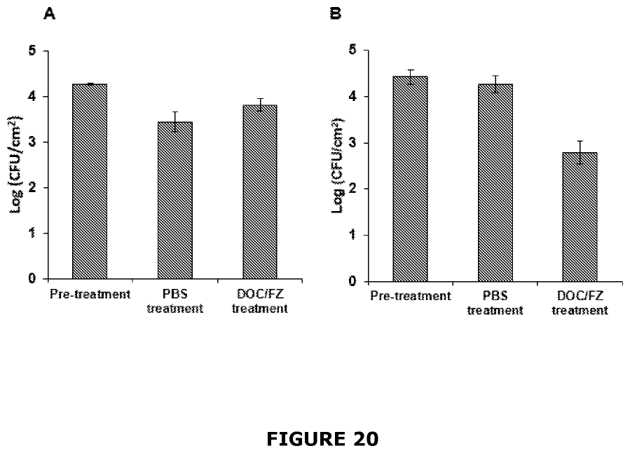

In another example, antibacterial efficacy of the DOC-FZ combination on E. coli-inoculated meat was examined, to assess the potential of the topical application of this combined therapy. In this experiment, E. coli-K1508-inoculated rump steak slices with a surface area of about 4 cm² were either sprayed with the PBS solution, acting as vehicle-treated samples, or with the combination of DOC 2500 μg/mL and FZ 0.32 μg/mL. The efficacy was evaluated by determining the number of viable cells on the meat surface after drug treatments under two different conditions, 10 min at room temperature and 2 hr. at 30° C. It was found that the post-exposure time of 10 min after the treatment with the DOC/FZ solution at room temperature was not sufficient to cause any antibacterial effect (FIG. 20A). By contrast, incubation of the meat samples at 30° C. for 2 hours after treatment resulted in a 1.6 log reduction in the cell count per cm² (FIG. 20B). This finding indicates that the DOC/FZ treatment could act efficiently to reduce E. coli abundance on meat surface, as indicative of muscle texture in the wound infections.

### Example 7

The Efficacy of DOC-FZ Treatment on E. coli-Inoculated Hide

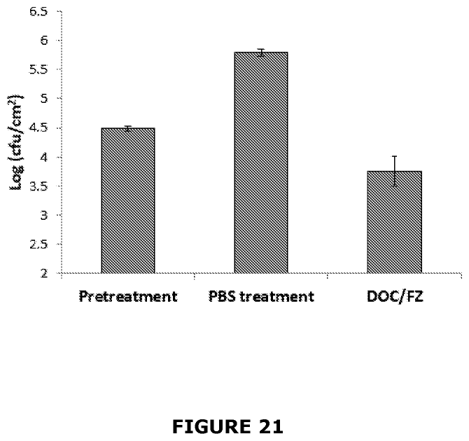

In another example, to further validate the potential topical use of the combination, the DOC/FZ treatment was also tested on cow hide. Particularly, pieces of cow hide (each with the total surface of 4.5 cm²) were inoculated with E. coli K12 strain K1508 and subsequently dipped into a solution containing 2500 μg/mL DOC and 0.32 μg/mL FZ for 6 h at 30° C. in comparison with water treatment as a vehicle control. The counts of viable cells before and after treatment were determined from the number of colony forming units on agar plates containing Streptomycin, to titer only the inoculated strain K1508 which is resistant to this antibiotic due to a mutation in the gene rpsL encoding for a ribosomal protein. This was necessary because the hide samples are always contaminated with an intrinsic large population of bacteria. In the water-treated hide sample the number of inoculated E. coli cells increased from 4.50 log CFU/cm² to 5.94 log CFU/cm² during 6 h of incubation, indicating that the cow hide contains sufficient amount of nutrients for bacteria to grow if left untreated (FIG. 21). By contrast, the DOC/FZ treatment not only inhibited cell growth but also decreased the E. coli viable cell count to 3.76 log CFU/cm² (equivalent to 80% reduction). The difference between the water-treated and FZ-DOC-treated cultures was 1.18 log CFU/cm² (equivalent to 94% reduction). The example here shows that the FZ-DOC treatment not only inhibits growth of E. coli but also decreases the viable cell count on the cow hide as a model for skin infection.

### Example 8

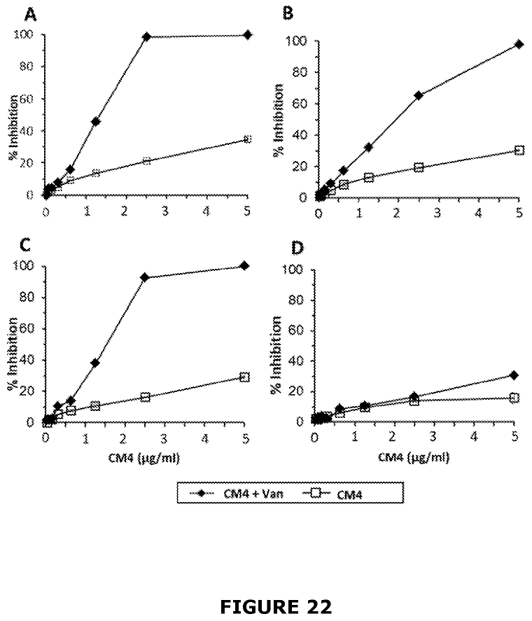

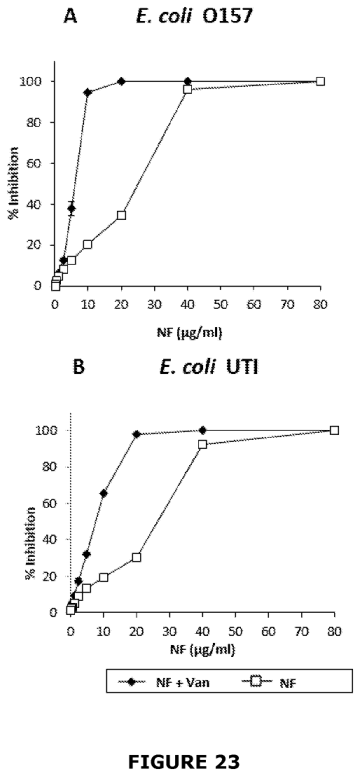

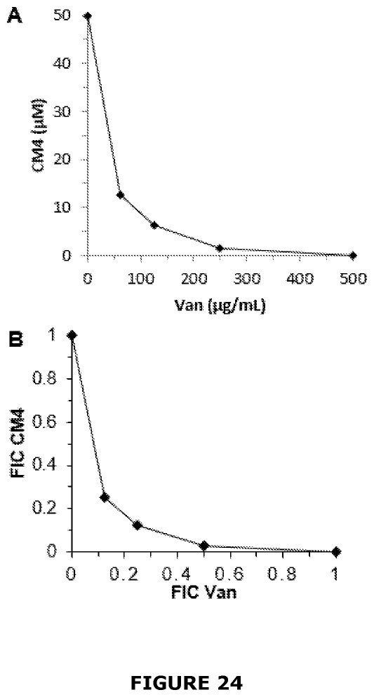

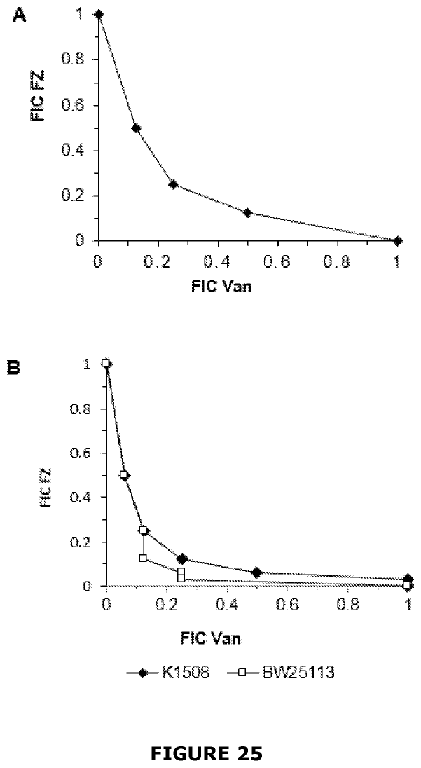

Another component of the three-way synergy between nitrofurans, bile salts and vancomycin is the nitrofuran-vancomycin synergy. The Van synergy with CM4, NF and FZ was shown for three Enterobacterial species, Escherichia coli (laboratory strains, an O157 isolate and a UTI isolate), Salmonella enterica SA223a and Citrobacter gillenii (FIG. 22-25). The isobolograms for the combinations of CM4 or FZ with Van acting on E. coli strains gave the FICi values between 0.125 and 0.375 (FIG. 24-25), which all fit in the synergy range (<0.5) (Doern, 2014).

### Example 9

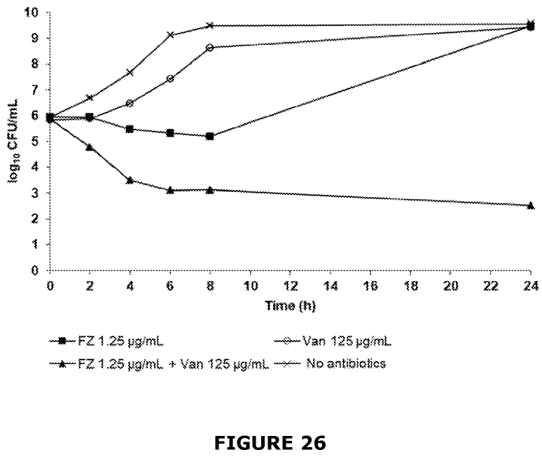

Bactericidal effect of antibacterials is required for elimination of bacteria from an infected individual or from inanimate objects such as catheters and implants, hence it is essential to confirm that the synergistic combination act through killing bacteria. To assay of synergistic killing effect by FZ and Van at concentrations that were sub-lethal when applied alone was carried out (FIG. 26). Particularly, the E. coli K12 laboratory strain BW25113 at a starting inoculum of about 10⁶ CFU/mL was exposed to Van 125 μg/ml and FZ 1.25 μg/ml. The titre of viable bacteria was monitored over the time-course of 24 h at 30° C. (FIG. 26). Each concentration was sub-inhibitory when the Van and FZ were used alone in growth inhibition assay of the same strain (FIG. 25B), but were inhibitory in combination.

After 24 h, the total cell count in the sample treated with the Van and FZ mixture was about six orders of magnitude lower than that in the sample where no antibiotics were applied (FIG. 26). By comparison, the viable titer of a control culture exposed to bacteriostatic antibiotic tetracycline did not decrease over the time-course of 24 h. The antibacterial interaction is interpreted as synergistic if the combinatorial treatment caused a killing efficiency 100-fold higher than the most active agent alone (Doern, 2014). Therefore, the synergy between Van and FZ was confirmed in killing E. coli K12 strain BW25113. Of another note is that the combinatorial treatment of Van and FZ reduced the total cell count by two orders of magnitude and can hence be considered bactericidal (FIG. 26).

### INDUSTRIAL APPLICABILITY

The antibacterial combinations and compositions of the invention find use as disinfectants, and in the treatment and prevention of microbial, particularly bacterial, infections, diseases and or conditions.

### REFERENCES

Baba, T., Ara, T., Hasegawa, M., Takai, Y., Okumura, Y., Baba, M., Datsenko, K. A., Tomita, M., Wanner, B. L. & Mori, H. 2006. Construction of Escherichia coli K-12 in-frame, single-gene knockout mutants: the Keio collection. Mol Syst Biol, 2, 2006 0008. Available: DOI 10.1038/msb4100050.
Begley, M., Gahan, C. G. & Hill, C. 2005. The interaction between bacteria and bile. FEMS Microbiol Rev, 29, 625-51. Available: DOI 10.1016/j.femsre.2004.09.003.
Bertenyi, K. K. & Lambert, I. B. 1996. The mutational specificity of furazolidone in the lad gene of Escherichia coli. Mutat Res, 357, 199-208.
Bollenbach, T. 2015. Antimicrobial interactions: mechanisms and implications for drug discovery and resistance evolution. Curr Opin Microbiol, 27, 1-9. Available: DOI 10.1016/j.mib.2015.05.008.
Burtis, C. A., Bruns, D. E., Sawyer, B. G. & Tietz, N. W. 2015. Tietz fundamentals of clinical chemistry and molecular diagnostics, St. Louis, Mo., Elsevier/Saunders.
Cherepanov, P. P. & Wackernagel, W. 1995. Gene disruption in Escherichia coli: TcR and KmR cassettes with the option of Flp-catalyzed excision of the antibiotic-resistance determinant. Gene, 158, 9-14.

Cremers, C. M., Knoefler, D., Vitvitsky, V., Banerjee, R. & Jakob, U. 2014. Bile salts act as effective protein-unfolding agents and instigators of disulfide stress in vivo. Proceedings of the National Academy of Sciences of the United States of America, 111, E1610-E1619. Available: DOI 10.1073/pnas.1401941111.
Doern, C. D. 2014. When does 2 plus 2 equal 5? A review of antimicrobial synergy testing. J Clin Microbiol, 52, 4124-8. Available: DOI 10.1128/JCM.01121-14.
Elyasi, S., Khalili, H., Dashti-Khavidaki, S. & Mohammadpour, A. 2012. Vancomycin-induced nephrotoxicity: mechanism, incidence, risk factors and special populations. A literature review. Eur J Clin Pharmacol, 68, 1243-55. Available: DOI 10.1007/s00228-012-1259-9.
Faustino, C., Serafim, C., Rijo, P. & Reis, C. P. 2016. Bile acids and bile acid derivatives: use in drug delivery systems and as therapeutic agents. Expert Opinion on Drug Delivery, 13, 1133-1148. Available: DOI 10.1080/17425247.2016.1178233.
Forrester, M. T. & Foster, M. W. 2012. Protection from nitrosative stress: a central role for microbial flavohemoglobin. Free Radic Biol Med, 52, 1620-33. Available: DOI 10.1016/j.freeradbiomed.2012.01.028.
Hajaghamohammadi, A., Safiabadi Tali, S. H., Samimi, R., Oveisi, S. & Kazemifar, A. M. 2014. Low dose furazolidone for eradication of H. pylori instead of clarithromycin: a clinical trial. Glob J Health Sci, 7, 235-9. Available: DOI 10.5539/gjhs.v7n1p235.
Iredell, J., Brown, J. & Tagg, K. 2016. Antibiotic resistance in Enterobacteriaceae: mechanisms and clinical implications. BMJ, 352, h6420. Available: DOI 10.1136/bmj.h6420.
Kitagawa, M., Ara, T., Arifuzzaman, M., Ioka-Nakamichi, T., Inamoto, E., Toyonaga, H. & Mori, H. 2005. Complete set of ORF clones of Escherichia coli ASKA library (a complete set of E. coli K-12 ORF archive): unique resources for biological research. DNA Res, 12, 291-9. Available: DOI 10.1093/dnares/dsi012.
Lederberg, J. 2000. Encyclopedia of microbiology, San Diego, Academic Press.
Levine, D. P. 2006. Vancomycin: a history. Clin Infect Dis, 42 Suppl 1, S5-12. Available: DOI 10.1086/491709.
Lewin, B., Krebs, J. E., Kilpatrick, S. T., Goldstein, E. S. & Lewin, B. 2011. Lewin's genes X, Sudbury, Mass., Jones and Bartlett.
Madigan, M. T., Madigan, M. T. & Brock, T. D. 2009. Brock biology of microorganisms, San Francisco, Calif., Pearson/Benjamin Cummings.
Marston, H. D., Dixon, D. M., Knisely, J. M., Palmore, T. N. & Fauci, A. S. 2016. Antimicrobial Resistance. JAMA, 316, 1193-1204. Available: DOI 10.1001/jama.2016.11764.
Mcosker, C. C. & Fitzpatrick, P. M. 1994. Nitrofurantoin: mechanism of action and implications for resistance development in common uropathogens. J Antimicrob Chemother, 33 Suppl A, 23-30.
Mergenhagen, K. A. & Borton, A. R. 2014. Vancomycin nephrotoxicity: a review. J Pharm Pract, 27, 545-53. Available: DOI 10.1177/0897190014546114.
Merritt, M. E. & Donaldson, J. R. 2009. Effect of bile salts on the DNA and membrane integrity of enteric bacteria. J Med Microbiol, 58, 1533-41. Available: DOI 10.1099/jmm.0.014092-0.
Meyers, R. A. 1995. Molecular biology and biotechnology: a comprehensive desk reference, New York, VCH.
Nishino, K. & Yamaguchi, A. 2001. Analysis of a complete library of putative drug transporter genes in Escherichia coli. J Bacteriol, 183, 5803-12. Available: DOI 10.1128/JB.183.20.5803-5812.2001.
O'neill, J. & Review-Team 2014. Antimicrobial resistance: tackling a crisis for the health and wealth of nations. In: Welcome Trust, H. G. (ed.) Review on Antimicrobial Resistance Available: https://amr-review.org/sites/default/files/AMR%20Review%20Paper%20-%20Tackling%20a%20crisis%20for%20the%20health%20and%20wealth%20off%20nations 1.pdf [Accessed 03-02-2017].
Ona, K. R., Courcelle, C. T. & Courcelle, J. 2009. Nucleotide excision repair is a predominant mechanism for processing nitrofurazone-induced DNA damage in Escherichia coli. J Bacteriol, 191, 4959-65. Available: DOI 10.1128/JB.00495-09.
Paul, S., Alegre, K. O., Holdsworth, S. R., Rice, M., Brown, J. A., Mcveigh, P., Kelly, S. M. & Law, C. J. 2014. A single-component multidrug transporter of the major facilitator superfamily is part of a network that protects Escherichia coli from bile salt stress. Mol Microbiol, 92, 872-84. Available: DOI 10.1111/mmi.12597.
Petri, W. A. 2005. Treatment of Giardiasis. Curr Treat Options Gastroenterol, 8, 13-17.
Reddy, C. A. 2007. Methods for general and molecular microbiology, Washington, D.C., ASM Press.
Roldan, M. D., Perez-Reinado, E., Castillo, F. & Moreno-Vivian, C. 2008. Reduction of polynitroaromatic compounds: the bacterial nitroreductases. FEMS Microbiol Rev, 32, 474-500. Available: DOI 10.1111/j.1574-6976.2008.00107.x.
Sambrook, J. & Russell, D. W. 2001. Molecular cloning: a laboratory manual (3rd. Edn.), Cold Spring Harbor, Cold Spring Harbor.
Sandegren, L., Lindqvist, A., Kahlmeter, G. & Andersson, D. I. 2008. Nitrofurantoin resistance mechanism and fitness cost in Escherichia coli. J Antimicrob Chemother, 62, 495-503. Available: DOI 10.1093/jac/dkn222.
Silver, L. L. 2011. Challenges of antibacterial discovery. Clin Microbiol Rev, 24, 71-109. Available: DOI 24/1/71 [pii] 10.1128/CMR.00030-10.
Singleton, P. & Sainsbury, D. 2006. Dictionary of microbiology and molecular biology, Chichester, West Sussex; Hoboken, N.J., Wiley.
Spagnuolo, J., Opalka, N., Wen, W. X., Gagic, D., Chabaud, E., Bellini, P., Bennett, M. D., Norris, G. E., Darst, S. A., Russel, M. & Rakonjac, J. 2010. Identification of the gate regions in the primary structure of the secretin pIV. Mol Microbiol, 76, 133-50. Available: DOI 10.1111/j.1365-2958.2010.07085.x.
Vumma, R., Bang, C. S., Kruse, R., Johansson, K. & Persson, K. 2016. Antibacterial effects of nitric oxide on uropathogenic Escherichia coli during bladder epithelial cell colonization—a comparison with nitrofurantoin. J Antibiot (Tokyo), 69, 183-6. Available: DOI 10.1038/ja.2015.112.
Whitby, L. G. & Whitby, L. G. 1993. Lecture notes on clinical biochemistry, Oxford; Boston, Blackwell Scientific Publications.
Who. 2017. WHO publishes list of bacteria for which new antibiotics are urgently needed. http://www.who.int/mediacentre/news/releases/2017/bacteria-antibiotics-needed/en/ [Online].
Yarlagadda, V., Manjunath, G. B., Sarkar, P., Akkapeddi, P., Paramanandham, K., Shorne, B. R., Ravikumar, R. & Haldar, J. 2016. Glycopeptide Antibiotic To Overcome the Intrinsic Resistance of Gram-Negative Bacteria. ACS Infect Dis, 2, 132-9. Available: DOI 10.1021/acsinfecdis.5b00114.
Zarkan, A., Macklyne, H. R., Chirgadze, D. Y., Bond, A. D., Hesketh, A. R. & Hong, H. J. 2017. Zn(II) mediates vancomycin polymerization and potentiates its antibiotic activity against resistant bacteria. Sci Rep, 7, 4893. Available: DOI 10.1038/s41598-017-04868-2.
Zarkan, A., Macklyne, H. R., Truman, A. W., Hesketh, A. R. & Hong, H. J. 2016. The frontline antibiotic vancomycin induces a zinc starvation response in bacteria by binding to Zn(II). Sci Rep, 6, 19602. Available: DOI 10.1038/srep19602.
Zhou, A., Kang, T. M., Yuan, J., Beppler, C., Nguyen, C., Mao, Z., Nguyen, M. Q., Yeh, P. & Miller, J. H. 2015. Synergistic interactions of vancomycin with different antibiotics against Escherichia coli: trimethoprim and nitrofurantoin display strong synergies with vancomycin against wild-type E. coli. Antimicrob Agents Chemother, 59, 276-81. Available: DOI 10.1128/AAC.03502-14.

What we claim is:

1. An antibacterial combination comprising a nitrofuran or a functional derivative or analogue thereof, and a bile salt or a functional derivative or analogue thereof.

2. The combination of claim 1 or claim 2, wherein the nitrofuran is selected from the group consisting of CM4, difurazone, furazolidone; nifurfoline, nifuroxazide, nifurquinazol, nifurtoinol, nifurzide, nitrofural (nitrofurazone), nitrofurantoin, ranbezolid and nifuratel, preferably wherein the nitrofuran is CM4, furazolidone or nitrofurantoin.

3. The combination of claim 1 or claim 2, wherein the bile salt or functional analogue or derivative thereof is selected from the group consisting of deoxycholate, cholate, chenodeoxycholate, taurocholate, glycocholate, taurochenodeoxycholate, glycochenodeoxycholate, lithocholate, and ursodeoxycholate.

4. The combination of any one of claims 1 to 3, wherein the bile salt or functional analogue or derivative thereof is sodium deoxycholate (DOC).

5. The combination of any one of claims 1 to 4, further comprising a glycopeptide antibiotic.

6. The combination of claim 5, wherein the glycopeptide antibiotic is vancomycin or a functional analogue or derivative thereof.

7. The combination of any one of claims 1 to 6 that inhibits the growth and/or proliferation of at least one Gram-negative bacterial species and/or that is bacteriostatic and or that is bactericidal for at least one Gram-negative bacterial species.

8. The combination of claim 7, wherein the at least one Gram-negative bacterial species is a species of Enterobacteriaceae.

9. The combination of claim 8, wherein the species of Enterobacteriaceae is selected from the group consisting of Escherichia spp., preferably E. coli; Salmonella spp., preferably S. enteritidis, S. infantis, S. dublin, S. typhimurium, S. paratyphi, S. schottmulleri, or S. choleraesuis; Citrobacter spp., preferably Citrobacter gillenii, Neisseria spp., preferably N. meningitis or N. gonorrhoeae; Acinetobacter spp.; Bacteroides spp., preferably B. fragilis; Bordetella spp., preferably B. pertussis or B. parapertussis; Brucella spp., preferably B. melitentis, B. abortus or B. suis; Campylobacter spp., preferably C. jejuni, C. coli or C. fetus; Enterobacter spp.; Haemophilus spp., preferably H. influenza or H. parainfluenzae; Legionella spp., preferably L. pneumophila; Pasteurella spp., preferably P. yersinia or P. multocida; Proteus spp., preferably P. mirabilis or P. vulgaris; Serratia spp., preferably S. marscences or S. liquifaciens; Shigella spp., preferably S. sonnei, S. flexneri, S. dysenteriae or S. boydii; Vibrio spp., preferably V. cholera or V. eltor; and Yersinia spp., preferably Y. enterolitica, Y. pseudotuberculosis or Y. pestis.

10. The combination of claim 9, wherein the species of Enterobacteriaceae is selected from the group consisting of Escherichia coli, Salmonella enterica and Citrobacter gillenii or a strain thereof.

11. A composition comprising a combination of any one of claims 1 to 10 and a carrier, diluent or excipient.

12. A pharmaceutical composition comprising a combination of any one of claims 1 to 10 or the composition of claim 11, and a pharmaceutically acceptable carrier, diluent or excipient.

13. A cosmetic composition comprising a combination of any one of claims 1 to 10 or the composition of claim 11, and a cosmetically acceptable carrier, diluent or excipient.

14. A method of inhibiting the growth and/or proliferation of at least one Gram-negative bacterial species and/or of killing at least one Gram-negative bacterial species comprising contacting the at least one Gram-negative bacterial species with the combination of any one of claims 1 to 10 or the composition of any one of claims 11 to 13.

15. The method of claim 14 wherein contacting comprises contacting an object or part thereof that comprises the at least one Gram-negative bacterial species.

16. The method of claim 15, wherein contacting is for a sufficient time to allow the combination or composition to inhibit the growth and/or proliferation of the at least one Gram-negative bacterial species on and/or in the object.

17. The method of claim 14 or claim 15, wherein the object is an inanimate object or part thereof, an animal or part thereof, or a plant or part thereof.

18. A method of treating a bacterial infection, disease or condition comprising administering a combination of any one of claims 1 to 10 or the composition of claim 11 or 12 to subject in need thereof.

19. The method of claim 18, wherein the bacterial infection, disease and/or condition is caused by, or is associated with at least one Gram-negative bacterial species, preferably wherein the Gram negative bacterial species is as defined in any one of claims 8 to 10.

20. The method of claim 18 or 19, wherein the subject is an animal or part thereof, preferably a mammal, preferably a human.

21. Use of a combination of any one of claims 1 to 10 or a composition of claim 11 in the manufacture of a medicament for treating a bacterial infection, disease or condition.

22. Use of a combination of any one of claims 1 to 10 or the composition of claim 11 as a disinfectant.

23. Use of a combination of any one of claims 1 to 10 or the composition claim 11 reduce or inhibit bacterial growth.

24. Use of a combination of any one of claims 1 to 11 or the composition of claim 11 to make a cosmetic.
